(19) United States
(12) Patent Application Publication
(10) Pub. No.: US 2026/0191913 A1
(43) Pub. Date: Jul. 9, 2026

(54) MITOCHONDRIAL TRANSPLANTATION AND USE THEREOF IN OCULAR DISEASES

(71) Applicant: Mor Research Applications Ltd., Ramat-Gan (IL)

(72) Inventors: Ziv Rotfogel, Kfar Saba (IL); Avital Eisenberg, Rehovot (IL)

(21) Appl. No.: 19/434,932

(22) Filed: Dec. 29, 2025

Related U.S. Application Data
(63) Continuation-in-part of application No. 18/364,319, filed on Aug. 2, 2023, now Pat. No. 12,508,282.
Application No. 18/364,319 is a continuation-in-part of application No. 17/594,141, filed on Apr. 3, 2020, now Pat. No. 12,478,644.
(60) Provisional application No. 62/828,704, filed on Apr. 3, 2019.

(51) Int. Cl. A61K 35/407; A61K 45/06; A61P 27/02; A61P 39/06

(52) U.S. Cl. CPC A61K 35/407; A61K 45/06; A61P 27/02; A61P 39/06

(57) ABSTRACT

Methods for treatment of damaged corneal endothelium are provided, applicable for treatment or protection of corneal endothelium in various circumstances such as oxidative stress, age-related decline in endothelial cell density, inherited or non-inherited degenerative disease such as Fuchs endothelial corneal dystrophy, surgical trauma, increased intraocular pressure (IOP) or contact lens overuse. Further provided are methods for treatment of retinal degeneration such as in diabetic retinopathy, age-related macular degeneration and glaucoma. The methods comprise the transplantation of viable isolated, exogenous, mitochondria systemically and/or directly to the cornea or the retina.

### CROSS-REFERENCE TO RELATED APPLICATIONS

The present application is a continuation-in-part of application Ser. No. 18/364,319, which becomes U.S. Pat. No. 12,508,282 on Dec. 30, 2025. Parent application Ser. No. 18/364,319 is a continuation-in-part of application Ser. No. 17/594,141, now U.S. Pat. No. 12,478,644, which was a 371 of international application No. PCT/IB2020/053216, which claimed the benefit of provisional application No. 62/828,704. The entire contents of each of the above-mentioned applications are hereby incorporated herein by reference for all purposes.

### FIELD OF THE INVENTION

The present disclosure relates to mitochondrial transplantation, particularly, but not exclusively, to ocular mitochondrial transplantation, for treatment of damaged corneal endothelium and retinal degeneration.

### BACKGROUND OF THE INVENTION

Located in the cell cytoplasm, mitochondria are known as the powerhouse of the cell, as they produce most of the cell's energy in the form of adenosine triphosphate (ATP), while simultaneously producing a small and manageable amount of harmful oxidative agents known as reactive oxygen species (ROS). Human mitochondria have a special circular, double-stranded genome (mtDNA) that encodes 13 proteins essential for the function of the oxidative phosphorylation system, which is composed of four respiratory-chain complexes (complexes I-IV) and ATP synthase.

Besides their indispensable role in ATP production, mitochondria also participate in other processes including iron metabolism, calcium homeostasis, heme synthesis, steroid biosynthesis and oxidant/antioxidant (redox) balance. When mitochondrial function is impaired, an oxidative stress may occur, e.g., due to increased production of ROS, which, in turn, may lead to damage in DNA, proteins and lipids in the cell, decreased production of ATP and activation of signaling pathways that may eventually lead to cell death.

Mitochondrial dysfunction has been established in the pathogenesis of many conditions such as, but not limited to, aging, Parkinson's disease, Alzheimer's disease, diabetes and some psychiatric diseases. Genetic mitochondrial diseases are related to mutations in the mtDNA or mutated mitochondrial proteins encoded by the nucleus. In fact, apart from classic mitochondrial diseases, there is evidence for mitochondrial dysfunction in almost all known diseases. For example, mitochondrial dysfunction has been shown to contribute to the pathogenesis of ischemic and traumatic insults, for example, in cardiac infarctions, stroke, traumatic brain injury and spinal cord injury. Mitochondrial dysfunction may also be mediated by environmental factors.

The visual system is one of the highest energy-depending and demanding systems. It is, therefore, highly susceptible to impaired energy supply caused by mitochondrial dysfunction. Indeed, ocular manifestation is a prominent clinical feature of, e.g., various genetic mitochondrial disorders. Oxidative stress-induced mtDNA mutations and otherwise mitochondrial dysfunction have been shown to contribute to the progression of common sight-threatening conditions such as glaucoma, age-related macular degeneration (AMD), diabetic retinopathy and ocular ischemic insults. Several reports have suggested that mitochondria dysfunction/impairment contributes to acute and chronic ischemia-induced retinal ganglion cells (RGCs) death in central retinal artery occlusion, ischemic optic neuropathy and acute angle closure glaucoma. Since ocular ischemic insults are highly prevalent, yet still untreatable, new therapeutic modalities are an unmet need.

Mitochondrial transplantation is a therapeutic strategy attempted for treatment of ischemic insults in the heart, liver and nervous system. In this treatment modality, mitochondria isolated from a healthy tissue, in vitro, are injected into damaged tissue. Injection of isolated mitochondria into the damaged, e.g., ischemic tissue, results in the internalization of the mitochondria by the distressed cells and protects them from cell death. It is speculated that a fresh reservoir of healthy mitochondria decreases ROS production within the cell, provides a new pool of intact, exogenous mtDNA and increases energy production and calcium buffering capacity. The internalized exogenous mitochondria may also provide the cell with enough energy for mitophagy of damaged mitochondria (a form of mitochondria-specific targeted autophagy), that also contributes to cell survival.

### SUMMARY OF THE INVENTION

One aspect of the present disclosure relates to methods for treating various ocular pathologies related to, or associated with, mitochondrial dysfunction, mitochondrial destruction and/or depletion in the eye of a subject, whereby a therapeutically effective amount of isolated, exogenous mitochondria are delivered to the diseased eyes of the subject. In some embodiments, a disclosed method is directed to the treatment of an ocular manifestation of a systemic disease, disorder or condition associated with mitochondrial dysfunction. Systemic diseases which may have ocular manifestation include, for example, congenital, vascular, neoplastic, autoimmune, idiopathic, infectious, metabolic/endocrine or hypertension diseases. A disorder or condition that may have ocular manifestation is, for example, trauma, drugs usage and/or exposure to toxins.

In some embodiments, a disclosed method is directed to the treatment of an ophthalmic disease, disorder or condition associated with mitochondrial dysfunction in a subject, such as, but not limited to, an ocular sequelae of a primary mitochondrial disease (resulting, e.g., from direct impairment of mitochondrial functions by mutations in nuclear DNA and/or mitochondrial DNA), aging, environmental factors, an injury to the eye, or a secondary mitochondrial dysfunction disease. Non-limiting examples of ophthalmic diseases that may be treated by a disclosed method include dominant optic atrophy (DOA), Leber hereditary optic neuropathy (LHON), chronic progressive external ophthalmoplegia (CPEO), pigmentary retinopathy, Kearns-Sayre syndrome (KSS), corneal clouding, diabetic retinopathy, glaucoma, age-related macular degeneration (AMD), proliferative vitreo-retinopathy (PVR), and Fuchs dystrophy.

In some embodiments, a disclosed method is directed to the treatment of an ocular ischemic insult in a subject, for example, retinal ischemic insult. Exemplary ischemic optic neuropathies that may be treated by a disclosed method include, e.g., non-arteritic anterior ischemic optic neuropathy (NAION) and central retinal artery occlusion (CRAO).

A disclosed method is further directed to the treatment of mechanical injuries, chemical injuries and/or dystrophies in the eye that may be alleviated or mitigated by mitochondrial replenishment and/or replacement. In some embodiments, treatment of a damaged or injured corneal epithelial layer is effected by mitochondrial transplantation.

Damage to the cornea epithelium may be caused by one or more of: a medicament, injury, a corrosive agent, exposure to a chemical or toxin, corneal abrasion, dry eye, a corneal dystrophy involving erosion, a neurotrophic disease causing incomplete lid closure, trichiasis, distichiasis, a restrictive eyelid disease, proptosis/exophthalmos, blepharoplasty, an ultraviolet burn, limbal stem cell deficiency, topical anesthetic abuse, neurotrophic keratopathy and infection. For example, chemical injuries may result, e.g., from exposure to a variety of substances such as acids, alkalis, and other toxins and corrosive agents. Alkali injuries, which are more common, may be caused by exposure to hazardous alkali such as sodium hydroxide (NaOH) that may significantly damage the eye, particularly the cornea.

Mitochondrial dysfunction is characterized, for example, by inadequate number of mitochondria, a dysfunction in mitochondria electron transport and ATP-synthesis machinery.

In some embodiments, treatment is effected by transplantation of isolated mitochondria. The transplanted mitochondria may be autologous, allogeneic, syngeneic or xenogeneic mitochondria.

In some embodiments, the transplanted mitochondria are freshly isolated mitochondria.

Mitochondria, in accordance with a disclosed method, may be transplanted via a route selected from direct injection or microinjection, topical application and/or systemic delivery. In some embodiments, mitochondria are administered directly to the eye, e.g., into ocular tissues, for example, by injection to one or more of: the vitreous, sub retina cornea, an ocular-muscle, the anterior chamber, or the suprachoroidal space. In some embodiments, mitochondria are topically applied to the surface of the eye (for example, by designated eye drops). In exemplary embodiments, the mitochondria are injected intravitreally.

In another aspect, the present disclosure relates to a formulation comprising a therapeutically effective amount of isolated mitochondria and a pharmaceutically acceptable carrier, for use in the treatment of mitochondrial dysfunction, mitochondrial destruction or depletion in the eye of a subject, and a disease, disorder or condition associated therewith. Diseases, disorders or conditions associated with mitochondrial dysfunction, mitochondrial destruction or depletion include, but are not limited to, ocular manifestation of systemic diseases or disorders, ophthalmic diseases, disorders or conditions, an ocular ischemic insult in a subject or chemical injuries.

In some embodiments, a disclosed formulation comprises fresh mitochondria.

In a further aspect, the present disclosure relates to a mitochondrial transplantation-based method for treatment of damaged endothelium of the cornea, whereby therapeutically effective amounts of exogenous, isolated mitochondria are delivered to damaged corneal endothelium cells. Corneal endothelium damage treatable by the disclosed method include, for example, damage caused by oxidative stress or Fuchs endothelial corneal dystrophy.

In some embodiments, mitochondrial transplantation is combined with administration of one or more modulators of MAPK pathways and/or the ROCK pathway, for example, agents that activate the ERK and/or JNK pathways, thereby, augmenting or improving the mitochondrial effect in corneal wound healing.

A further aspect of the present disclosure relates to treating retinal degeneration, whereby exogenous, isolated mitochondria are transplanted to the retinal layer, for example, into degenerated photoreceptors, degenerated retinal ganglion cells, degenerated and/or retinal pigment epithelium (RPE) cells.

A disclosed method of treatment can be applied at different stages of a retinal degenerative disease. For example, transplantation of exogenous isolated mitochondria can be applied for prevention, early intervention, or slowing the progression of the disease, as well as for ameliorating, improving or curing advanced or late-stage retinal degenerative disease.

In some embodiments, the retinal degeneration is not directly associated with mitochondrial dysfunction.

In some embodiments, retinal degeneration is caused by diabetic retinopathy or glaucoma.

In some embodiments, any of the disclosed methods comprises transplantation of preserved mitochondria.

In yet another aspect, the present disclosure relates to a kit comprising (a) isolated mitochondria or a formulation comprising same; (b) means to administer the isolated mitochondria to a subject in need thereof; and optionally (c) written instructions.

In some embodiments, a contemplated kit comprises freshly isolated or preserved mitochondria.

Further embodiments and the full scope of applicability of the present disclosure will become apparent from the detailed description given hereinafter. However, it should be understood that the detailed description and specific examples, while indicating preferred embodiments of the disclosure, are given by way of illustration only, since various changes and modifications within the spirit and scope of the invention will become apparent to those skilled in the art from this detailed description.

### BRIEF DESCRIPTION OF THE FIGURES

The patent or application file contains at least one drawing executed in color. Copies of this patent or patent application publication with color drawings will be provided by the Office upon request and payment of the necessary fee.

Some embodiments of the present disclosure are herein described, by way of example only, with reference to the accompanying drawings. With specific reference now to the drawings in detail, it is stressed that the particulars shown are by way of example and for purposes of illustrative discussion of embodiments disclosed herein. In this regard, the description taken with the drawings makes apparent to those skilled in the art how embodiments described herein may be practiced.

### IN THE DRAWINGS

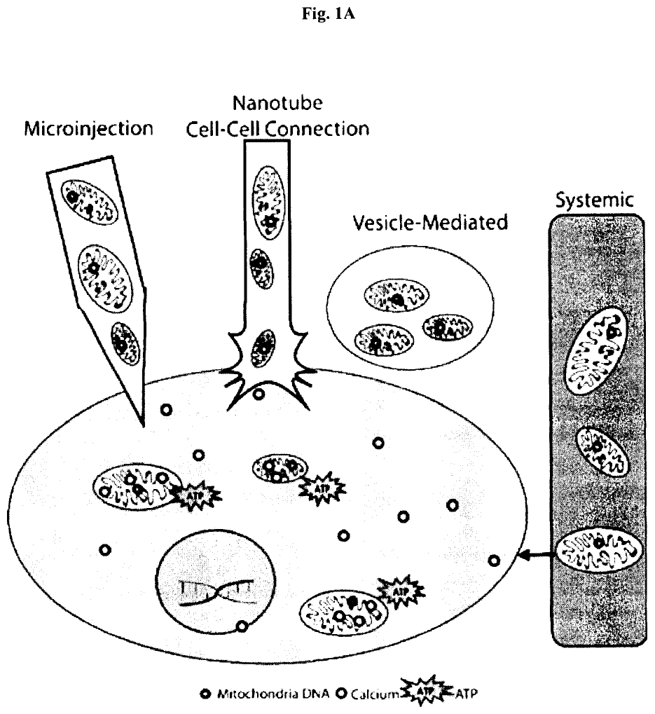

FIGS. 1A-1C are schemes illustrating various exogenous mitochondria delivery techniques and routes. FIG. 1A illustrates microinjection, nanotube cell-cell connection, vesicle-mediated delivery and systemic injection (Gollihue and Rabchevsky, 2017, Mitochondrion; 35:70-79). FIG. 1B is a schematic presentation of the eye and of intravitreal injection site (Asai et al., 2013, Int J. Nanomedicine, 8:495-503). FIG. 1C is a background art schematic presentation of further optional intraocular sites for mitochondrial injection (Dubald et al., 2018, Pharmaceutics, 13:10);

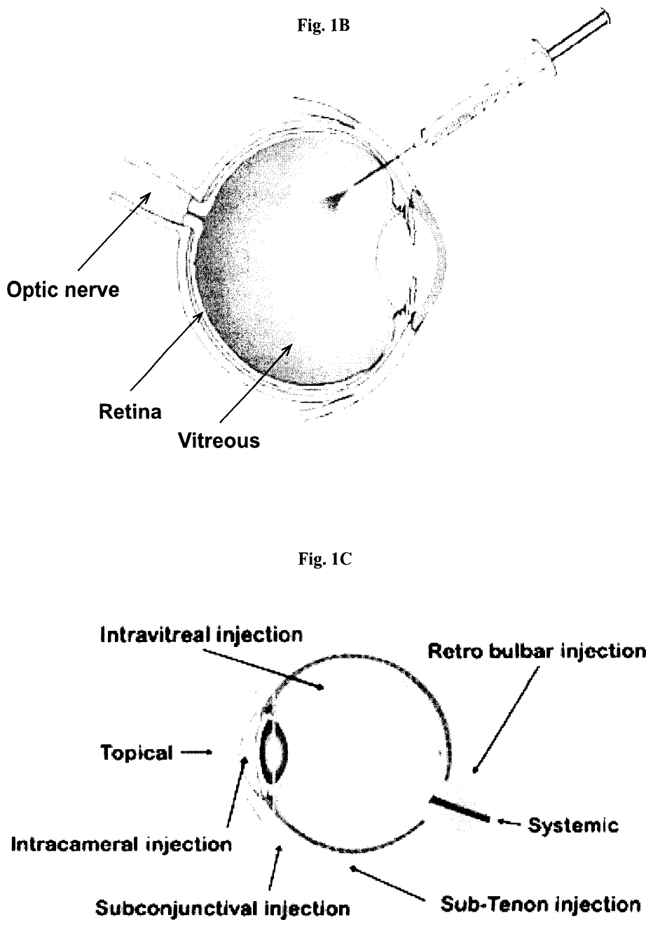

FIGS. 2A-2B are images of various layers of the retina. FIG. 2A is an optical coherence tomography (OCT) image of retinal layers, showing the location of the retinal ganglion cells layers in contact with the vitreous. RNFL/GC: retinal nerve fiber layer/ganglion cells layer; INL: inner nuclear layer; ONL: outer nuclear layer; IS/OS: junction between photoreceptors inner/outer segments; RPE: retinal pigment epithelium. FIG. 2B is a background art scheme showing a section of the retina and the optic nerve (https://biology4isc.weebly.com/eye.html);

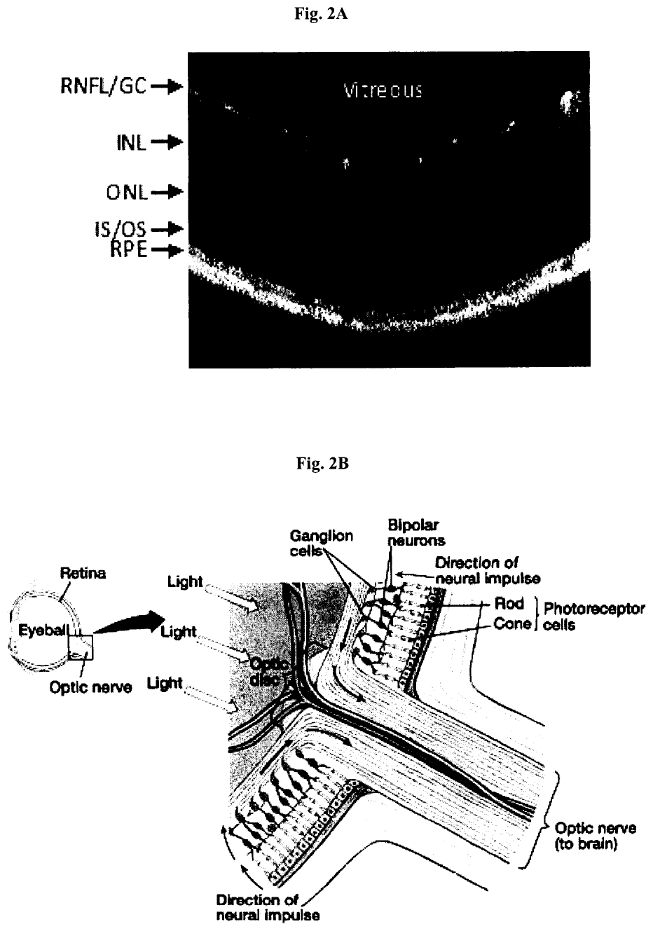

FIGS. 3A-3D show purification and quality of mitochondria isolated from mouse liver. FIG. 3A is a Western blot analysis showing the mitochondrial marker Cox4 and the cytosolic marker β-actin in a purified mitochondrial fraction (Mito), a cytosolic fraction (Cyto) and a control sample taken before fractionation (Input). FIG. 3B is a graph showing oxygen level changes following addition of mitochondrial substrates (succinate, ADP) to purified mitochondria. FIG. 3C is a bar graph showing base-line level of ATP production in purified mitochondria (Mito), and ATP levels following addition of ADP substrate (Mito-ADP) or the ATP-synthase inhibitor oligomycin (Mito-Olm). FIG. 3D is a collection of microscopic images of purified mitochondria stained with MitoTracker® Red CMXRos dye, a marker for active mitochondria with appropriate membrane potential (lower left image (red)), and with MitoTracker® Green FM dye, a marker for pan mitochondria (upper right image (green)). The lower right image is a superposition of the two former images, and the upper left image is a confocal image;

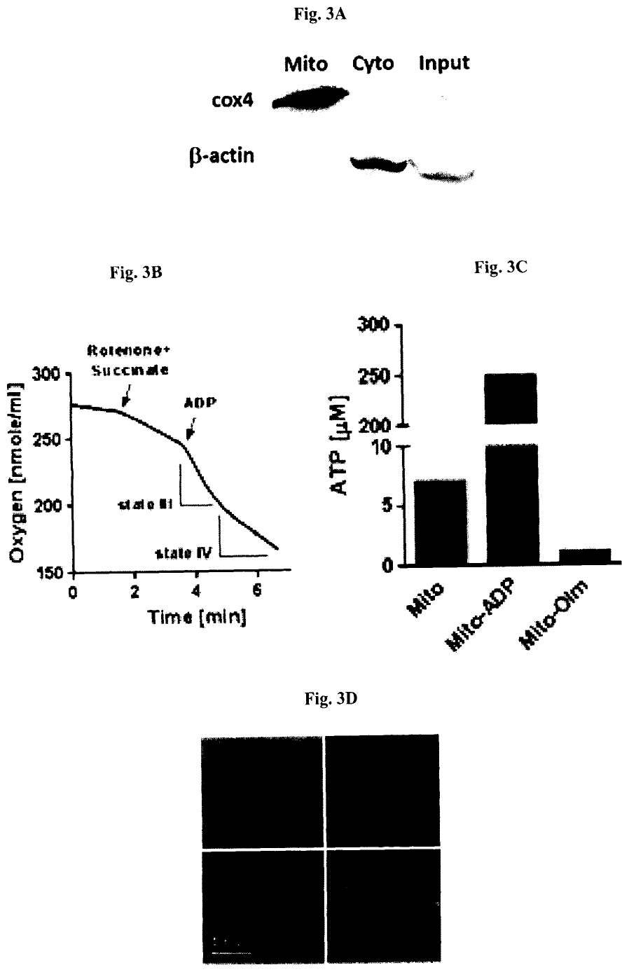

FIGS. 4A-4D show in vitro uptake by SH-SY5Y cells, human ARPE-19 cells, human 0cells (hCEC), and mouse RGC cells (661W) of mitochondria isolated from ARPE-19 cells or from mouse liver. FIG. 4A depicts flow cytometry histograms showing the counts of SH-SY5Y cells transplanted with exogenous mitochondria and pre-stained with MitoTracker® Green Fixable Mitochondrion dye (MTG FM) (middle histogram, Neurons+mito), unstained SH-SY5Y cells not transplanted with exogenous mitochondria (left histogram, Neurons; negative control), and SH-SY5Y cells non-transplanted with exogenous mitochondria and stained with MTG FM for endogenous mitochondria (right histogram, Stained neurons; positive control). FIG. 4B is a collection of immunofluorescence images showing uptake by SH-SY5Y cells of freshly isolated mitochondria stained with MitoTracker® Red CMXRos (MTR) (Mito; upper left image (red spots). Neuronal cells were immunostained for cytosolic, non-mitochondrial protein-actin (Actin; upper middle image (green)) and for Cox4 for marking endogenous mitochondria (Cox4; lower middle image (light blue)), and their nuclei were stained with the nuclear marker DAPI (DAPI; blue). The arrow in the merged image (lower right image) shows translocation of exogenous mitochondria into axons in a neuronal process. The lower left panel is a confocal image of stained neuronal cells. FIG. 4C is a collection of immunofluorescence images of SH-SY5Y cells transplanted with fresh exogenous mitochondria. The images depict exogenous mitochondria (red) and endogenous mitochondria (brighter spots (yellow). FIG. 4D is a bar graph showing mean green fluorescence intensity in flow cytometry analysis of 661W, hCEC and aRPE-19 cells transplanted with 1 million (1M) mitochondria stained with MTG;

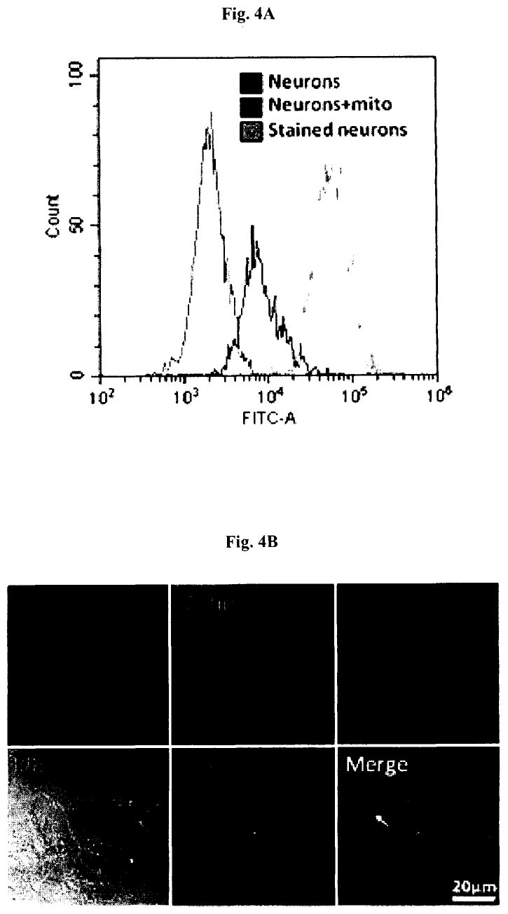

FIGS. 5A-5C show dose dependency of exogenous mitochondrial uptake. FIGS. 5A and 5B are flow cytometry histograms of hCEC cells and 661W cells, respectively, incubated for 24 hours with increasing amounts of labeled mitochondria. FIG. 5A: left to right histograms: control (unstained, non-transplanted cells); 200,000 mitochondria; 1M mitochondria; and five million (5M) mitochondria. FIG. 5B: left to right histograms: control (unstained, non-transplanted cells); half a million (0.5M) mitochondria; 1M mitochondria; and two million (2M) mitochondria. FIG. 5C is a bar graph showing the mean green fluorescence intensity relative to control obtained from 3 independent experiments;

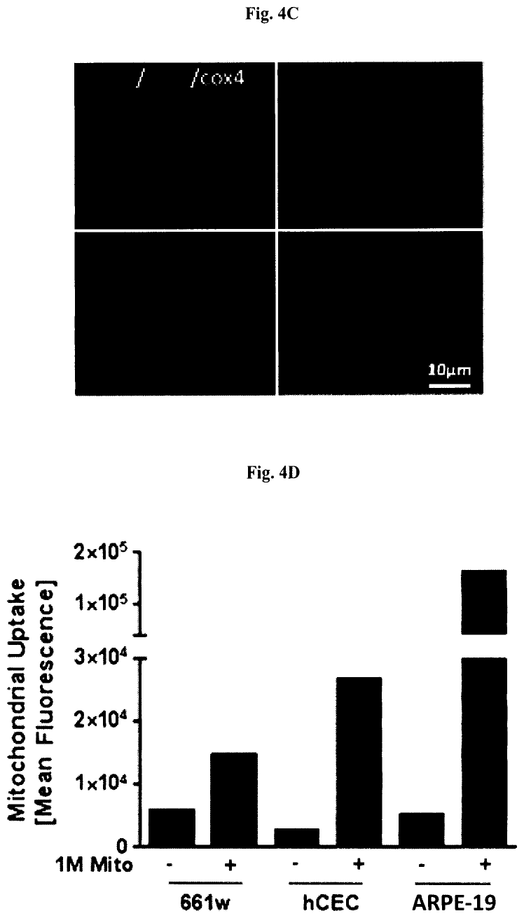

FIG. 6 is a bar graph showing ATP production in mouse RGC 661W cell line transplanted with fresh mitochondria isolated from C57BL/6 mice livers (n=5, i.e., 5 independent experiments);

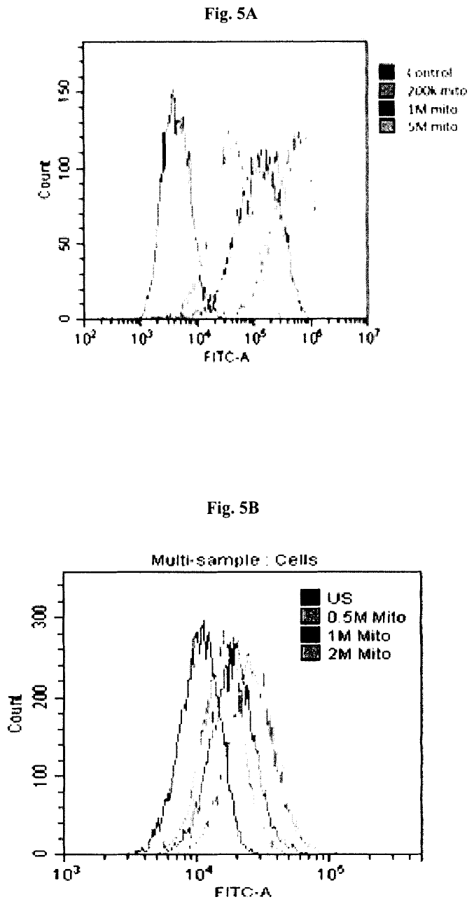

FIG. 7 is a bar graph showing the effect of oxidative stress (application of H₂O₂ for 1 hour) on mitochondrial uptake by mouse retinal ganglion cells (661W);

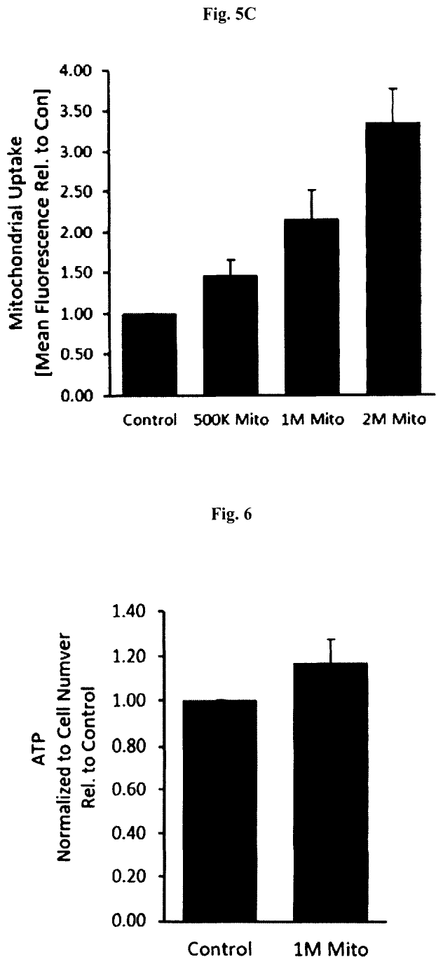

FIGS. 8A-8C show in vivo retinal uptake of purified, fresh mitochondria (about 10⁶ or about 10⁷) isolated from mouse liver. The mitochondria, stained with MitoTracker® Red CMXRos (Mito; red), were injected into the mouse vitreous of one eye, and the control eye was injected with PBS. FIG. 8A is a collection of confocal images (upper images) and fluorescent microscope images (lower images) of wholemount retinae injected either with stained mitochondria or PBS. FIGS. 8B-8C are collections of fluorescence microscope images of wholemount retinae of two exemplary mice injected with stained mitochondria. Middle images depict fluorescent internalized mitochondria (Mito; red); left images depict fluorescent ganglion cells immunostained with Tuj1 (TUJ1; green); and right images are merges of the left and middle images;

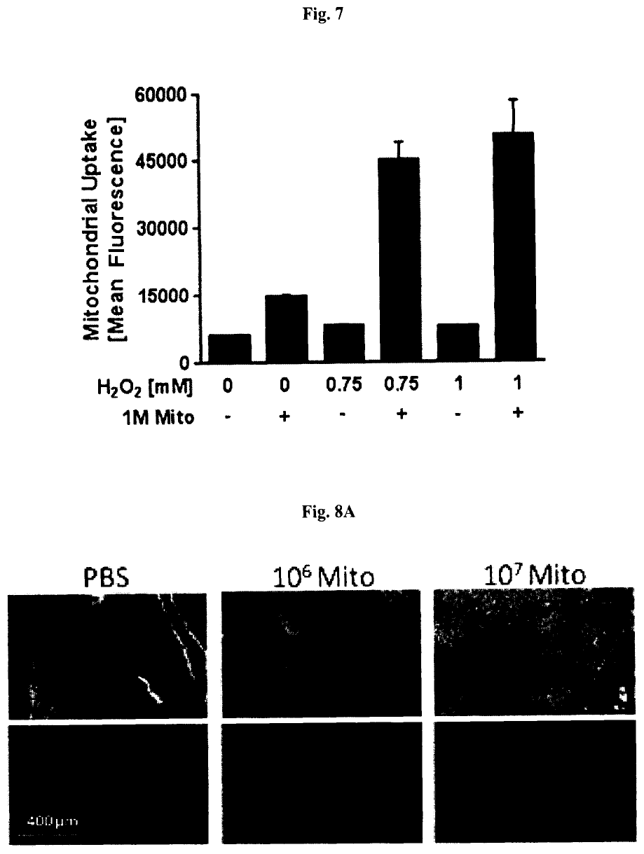

FIGS. 9A-9B are images of wholemount retinae of mice intravitreally injected with isolated mitochondria stained with MitoTracker® Red CMXRos (Mito; lower images (red)). FIG. 9A are confocal image (upper) and fluorescence image (lower) of retina transplanted with stained mitochondria injected to the vitreous of the control eye of an exemplary mouse, and FIG. 9B are confocal image (upper) and fluorescence image (lower) of retina transplanted with stained mitochondria injected immediately after optic nerve crush (ONC);

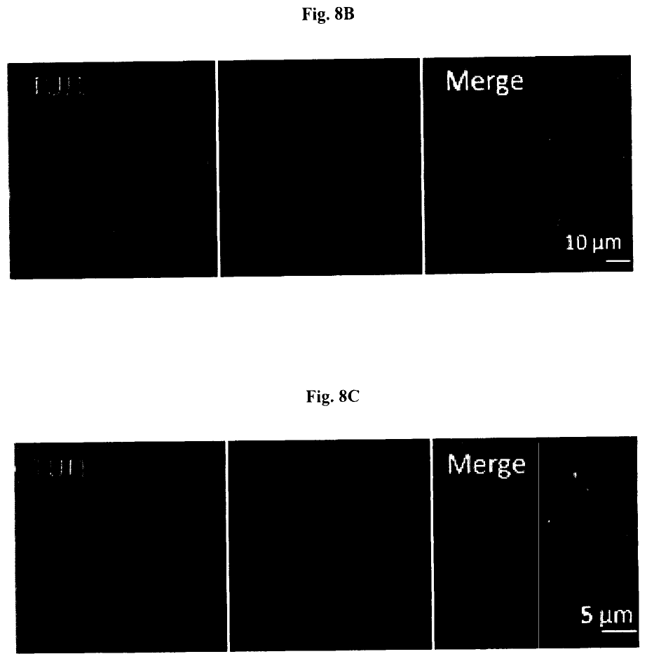

FIG. 10 is a collection of fluorescent microscope images of paraffin-embedded sections of an exemplary mouse eye intravitreally injected with freshly isolated mitochondria stained with MitoTracker® Red CMXRos (MTR), immediately after ONC. In the middle panel, the left image is DAPI staining (blue) of retinal layers. Retinal layers are indicated (ONL—outer nuclear layer; INL—inner nuclear layer; GCL—ganglion cells layer); the middle image depicts stained mitochondria in the retinal layers (red); and the left image is a superposition (merge) of the left and middle images. The rectangular frames in the images of the middle panel are zoomed-in and depicted in the upper panel (frame 1) and lower panel (frame 2), correspondingly, namely, from left to right: DAPI staining, MTR staining and merged zoomed-in images;

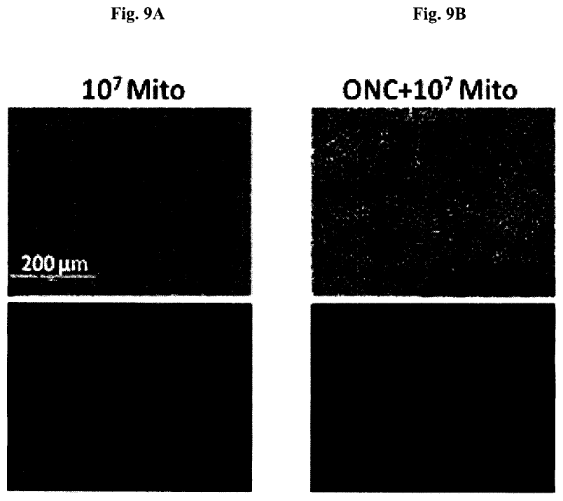

FIGS. 11A-11D show RGCs counts in exemplary sections of mice retinae observed two weeks after the mice experienced ONC. FIGS. 11A-11C are images of representative sections of H&E stained retina either intact or non-injured, namely, without ONC (FIG. 11A; untreated), injured (FIG. 11B; ONC) or injured and immediately treated intravitreally with isolated mitochondria (FIG. 11C; ONC+10⁷ Mito). The arrows point to the RGCs layer. FIG. 11D is a bar graph showing the number of intact RGCs in these sections;

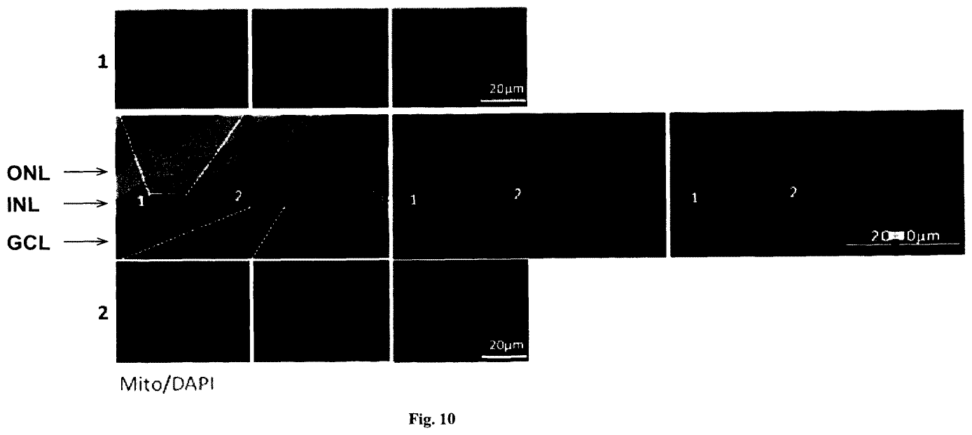

FIGS. 12A-12B are bar graphs showing the viability (measured as optical density (OD)) of two human cell lines of occular origin: hCEC (FIG. 12A) and ARPE-19 (FIG. 12B) subjected to oxidative stress, namely, treated with 2.5 μM H₂O₂ or 250 μM H₂O₂, respectively, followed by incubation with about 100,000 (100 K) fresh mitochondria (Mito) isolated from ARPE-19 cells. Cell viability was evaluated using neutral-red (for hCEC cells) or XTT (for ARPE-19 cells) assays;

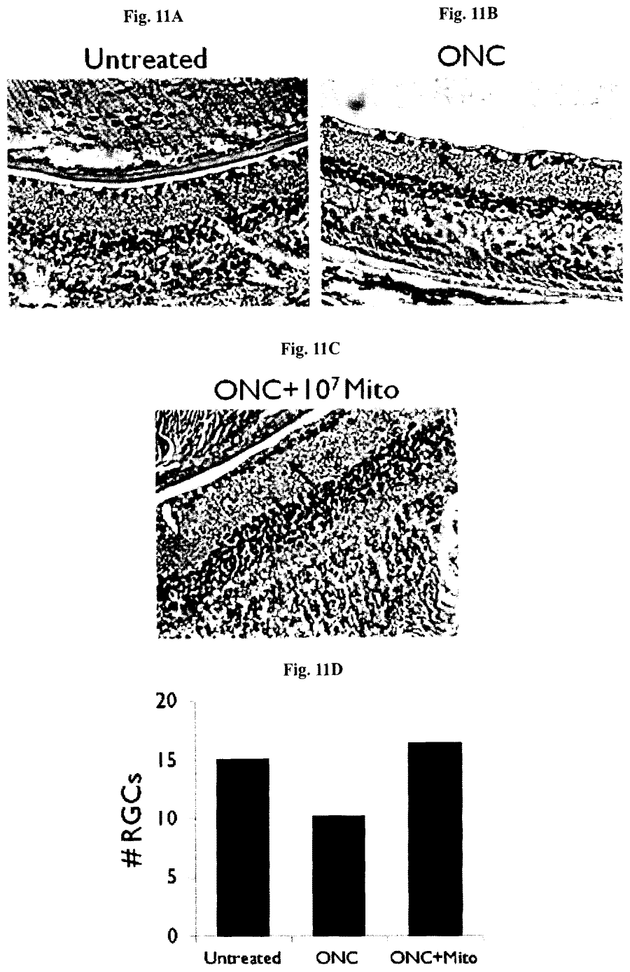

FIG. 13A-13B show the effect of mitochondrial transplantation on corneal wound healing. FIG. 13A are slit-lamp images and fluorescein staining of mice eyes injured by exposure to NaOH. Upper row: alkali injured eye not treated with mitochondrial transplantation. Lower row: alkali injured eye treated with mitochondria transplantation for 4 days. FIG. 13B are graphs showing epithelium wound size closure as a function of time from day 1 to day 4 after injuring the eye, for control (untreated eyes; upper graph) and eyes treated with mitochondria transplantation (lower graph). N=8 in control and mitochondria treated groups. **p<0.01; ****p<0.0001, Student's t-test;

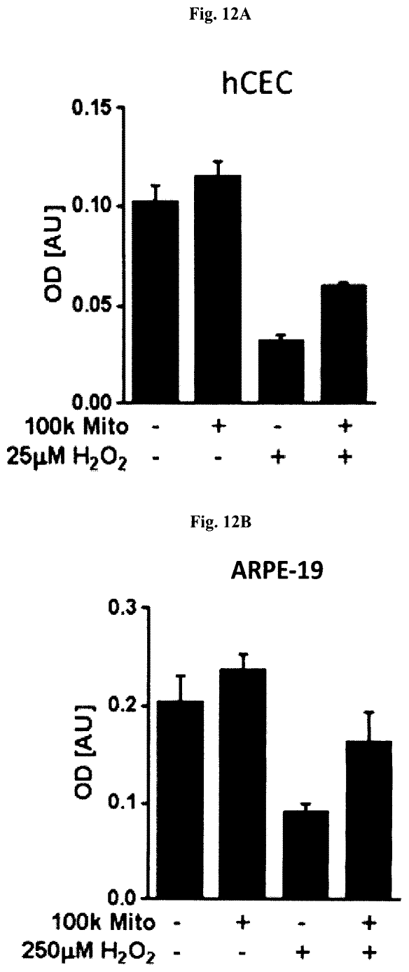

FIGS. 14A-14B show the effect of mitochondrial transplantation on corneal thickness. FIG. 14A are representative histology H&E-stained sections of corneas from alkali-injured eyes of mice either not treated with mitochondrial transplantation (control) or treated with mitochondria transplantation (Mito). Scale bar=200 μM. p<0.01, Student's t test. FIG. 14B are bar graphs showing the corneal thickness of mitochondria treated versus untreated eyes;

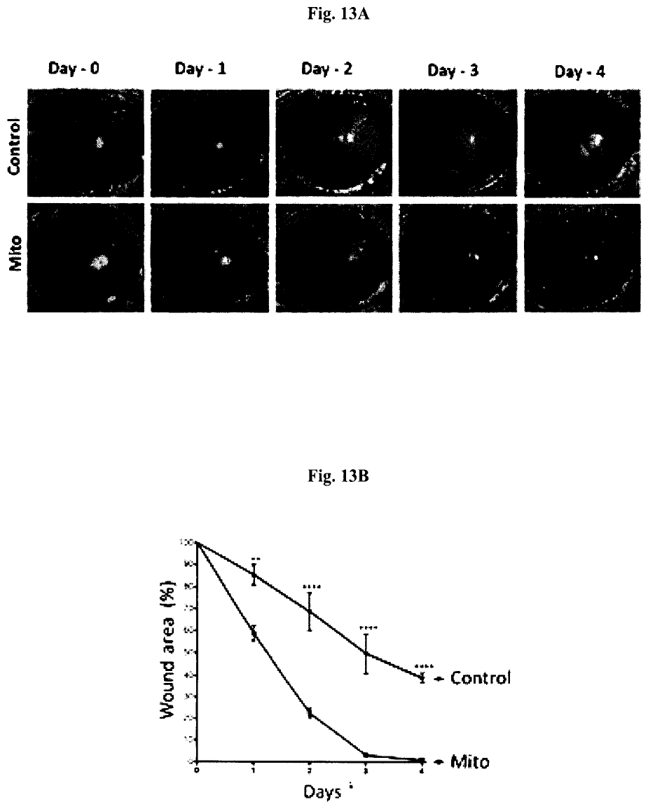

FIG. 15A-15B show the effect of mitochondrial transplantation on corneal wound healing in primary human corneal epithelial cells, isolated from human corneas. FIG. 15A are representative images of primary human corneal epithelial cell culture after a cross-shaped scratch was performed in the culture.

Left: Time 0h—immediately after the scratch was performed. Right: Time 24h-24 hours after scratching. Upper panel: control—cells treated with mitochondria transplantation after culture scratching. FIG. 15B are bar graphs quantifying the initial wound area after the scratch (top) and the percentage of wound closure, as determined by calculating the wound area at time 24h relative to the wound area at time 0h (Bottom). n=12 (control); n=18 (mito), seeded from a mix of cells harvested from 6 human corneas in 2 individual experiments. Scale bar=200 μM. ****p<0.0001, Student's t-test;

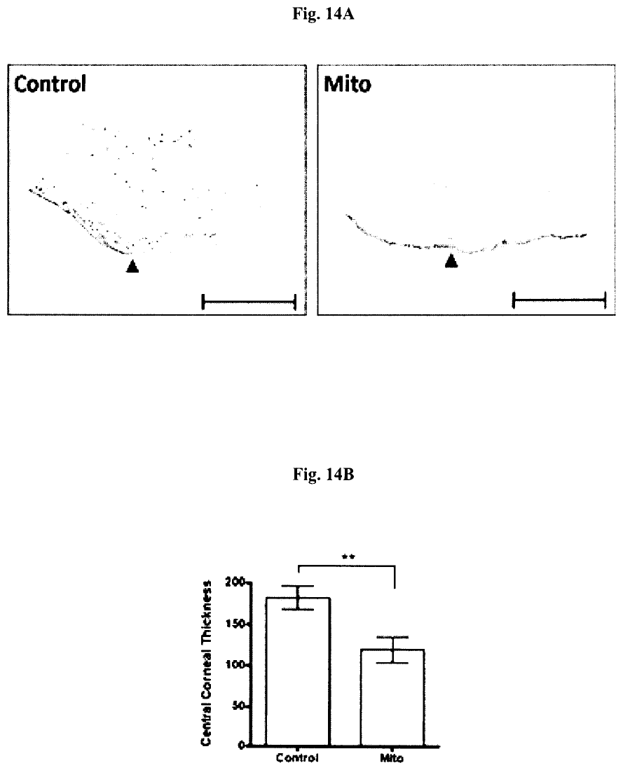

FIGS. 16A-16B are bar graphs showing the effect of mitochondria transplantation on proliferation of primary human epithelial cells as measured by counting ki67 positive cells (left) or Neutral Red uptake (right) in cells not treated with mitochondria (control) compared to cells treated with mitochondria transplantation (mito). *p<0.05, **p<0.01, Student's t-test;

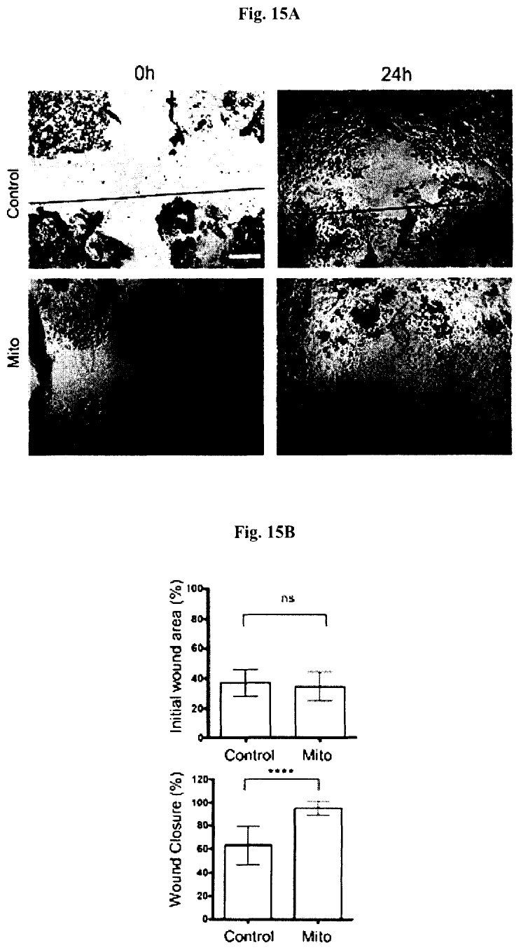

FIGS. 17A-17B show the effect of mitochondrial transplantation on migration of the human corneal endothelial cell line hCEC-12. Confluent monolayers of hCEC-12 cells were scratched in a cross pattern, and exogenous mitochondria (4 million or 20 million) were added to the culture medium on Day 0. “Wound” closure was evaluated 24 hours later (Day 1). Control cultures received no exogenous mitochondria. FIG. 17A is a collection of images of representative scratched cultures, with computational analysis used to automatically identify and measure cell-free regions (pink) and cell-covered regions (blue). FIG. 17B is a bar graph quantifying the remaining wound area, with statistical significance determined by Student's t-test (P<0.0001);

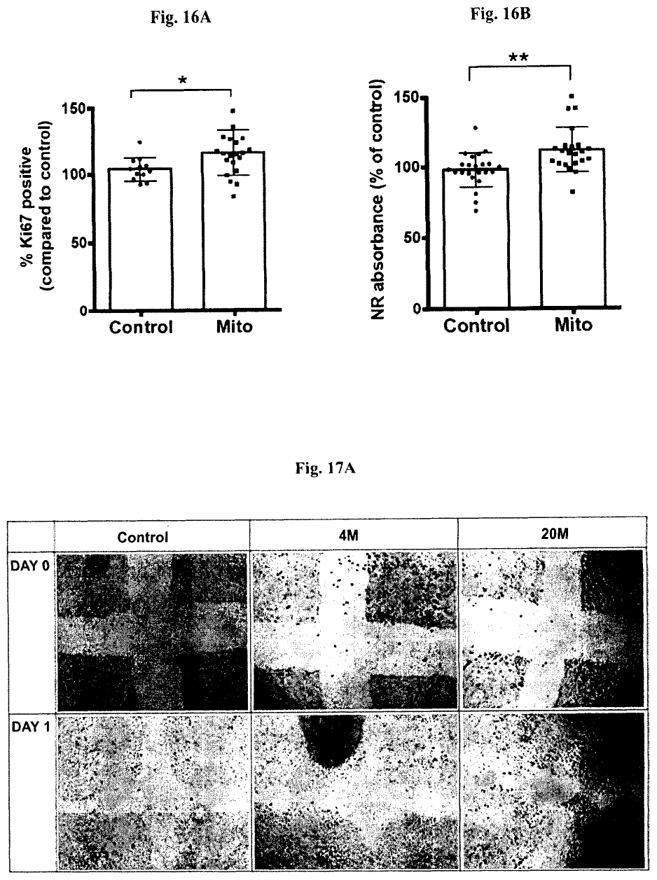

FIGS. 18A-18B show the effect of mitochondrial transplantation on the migration of primary human corneal endothelial cells. Primary human corneal endothelial cells were isolated from harvested human corneas and cultured. Confluent monolayers of cells were scratched in a cross pattern, and exogenous mitochondria (5 million) were added to the culture medium. “Wound” closure was evaluated 1, 2, 3 and 4 days later. Control cultures received no exogenous mitochondria. FIG. 18A are photographs of representative control and mitochondria-treated cultures, 4 days after scratching. FIG. 18B is a graph presenting the wound size reduction (% of original) as a function of time. Statistical significance determined by Student's t-test (P<0.0001);

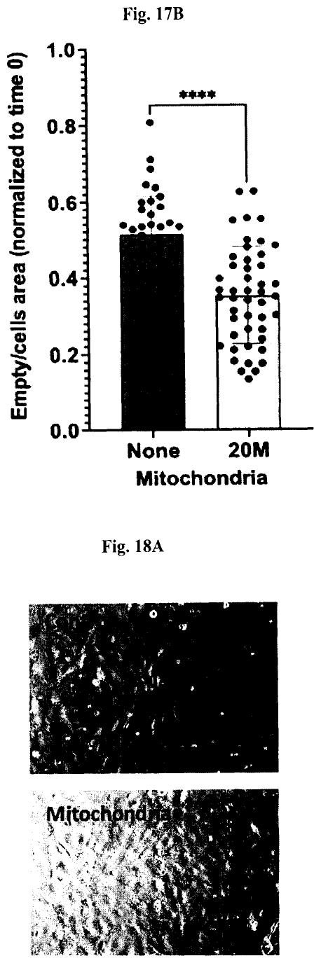

FIG. 19 is a bar graph showing the effect of mitochondria supplementation to primary human corneal endothelial cells culture on cell density. Primary human corneal endothelial cells were isolated from harvested human corneas and cultured to form sparse cultures. Exogenous mitochondria (5 million) were added to the culture medium of test cultures (“Mito”), whereas control cultures received no exogenous mitochondria (“Control)”. Cells were counted on Day 4. Data are presented for two individual experiments with one control and one test group in each, wherein cells were isolated from different corneas and treated with individual mitochondria isolations. Forty randomly selected cell fields were counted in each experiment. Two-tailed t-test was applied, p<0.0001;

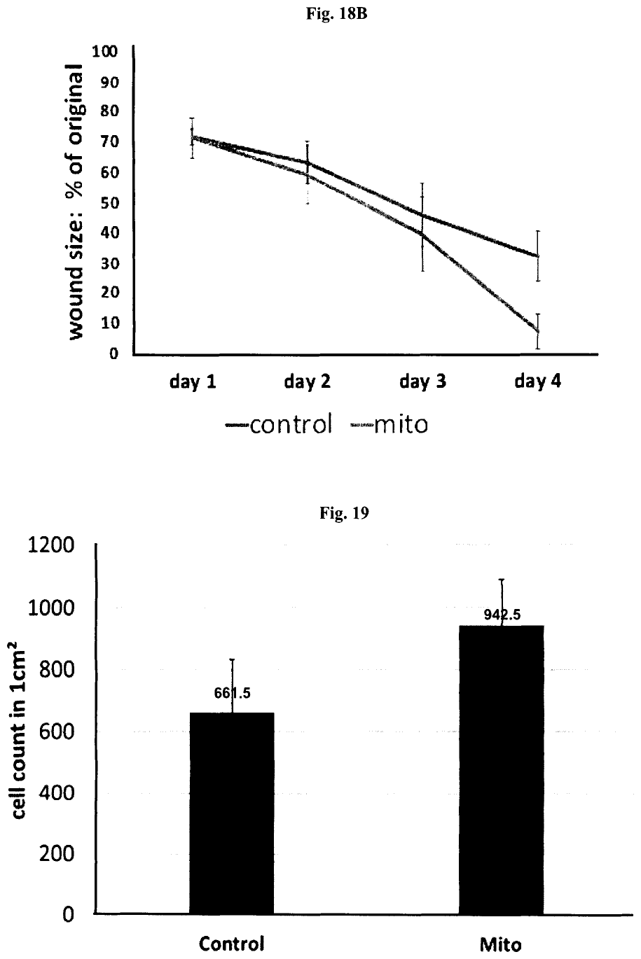

FIG. 20 is a bar graph depicting the effect of mitochondrial transplantation on ATP production in human corneal endothelial cells (hCEC-12). Cells were incubated with isolated mitochondria (Mito), while control cells received no added mitochondria. ATP levels were quantified using the ATPlite™ luminescence assay and analyzed by Student's t-test (*p<0.05);

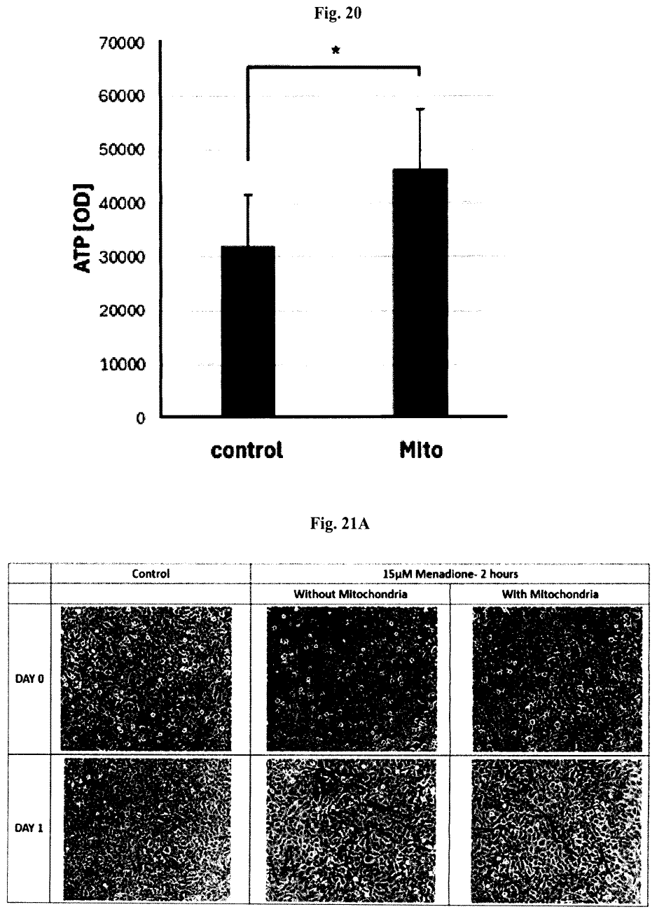

FIGS. 21A-21B show the impact of mitochondrial transplantation on oxidative stress-induced cellular reorganization in hCEC-12 cell culture. Human CEC-12 cultures were exposed to 15 μM menadione in serum-free medium (for inducing oxidative stress) for two hours, followed by supplementation with either isolated mitochondria (20 million) or media alone (control). FIG. 21A shows representative images of the cell cultures at Day 0 (basal state) and Day 1 (24 hours after mitochondrial treatment), with red arrows indicating rosette-shaped cellular clusters. FIG. 21B is a bar graph quantifying the rosette area. P<0.01;

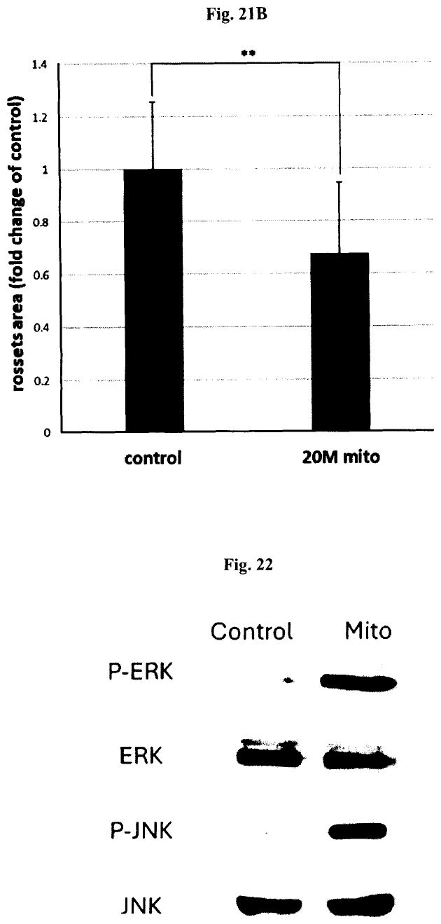

FIG. 22 shows a Western blot analysis of four distinct proteins of hCEC-12 lysates. Human CEC-12 were incubated for 3 hours with either isolated mitochondria or medium alone (control), and protein lysates were analyzed with antibodies against the total levels of extracellular signal-regulated kinase (ERK) and c-Jun N-terminal kinase (JNK) and their active phosphorylated forms (P-ERK and P-JNK, respectively);

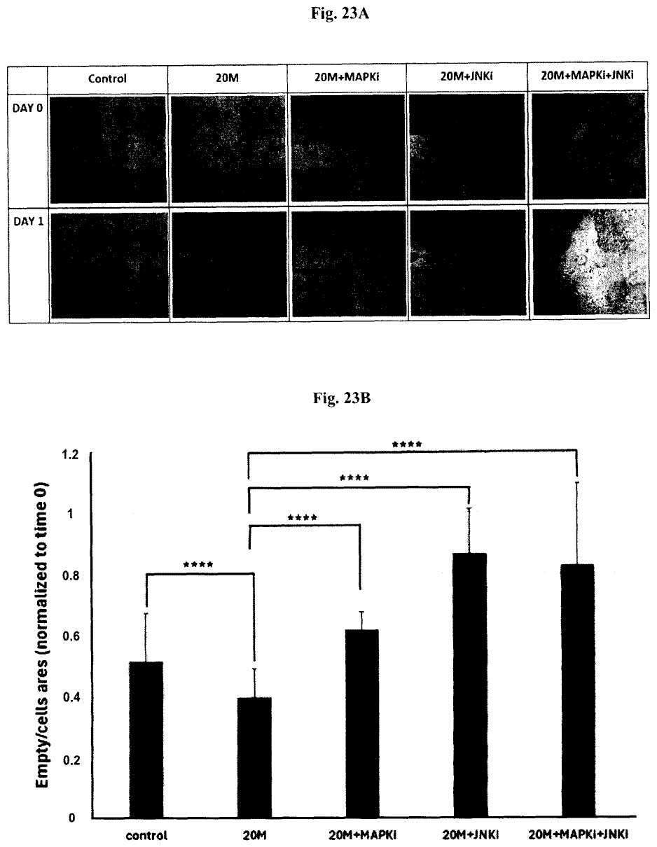

FIGS. 23A-23B show the effect of ERK and JNK activation on mitochondria-induced migration in corneal endothelial cells. Confluent monolayers of hCEC-12 cells were scratched in a cross pattern and then pre-treated for 1 hour with either MEK or JNK inhibitors (MAPKi and JNKi, respectively), both inhibitors together (MAPKi+JNKi), or DMSO (Control). Then, about 20 million (20M) exogenous mitochondria were added to the culture medium on Day 0. “Wound” closure was evaluated 24 hours later (Day 1). Control cultures received no exogenous mitochondria. FIG. 23A is a collection of images of representative scratched cultures, with computational analysis used to automatically identify and measure cell-free regions (pink) and cell-covered regions (blue). FIG. 23B is a bar graph quantifying the remaining wound area, with statistical significance determined by Student's t-test (****p<0.0001) ;

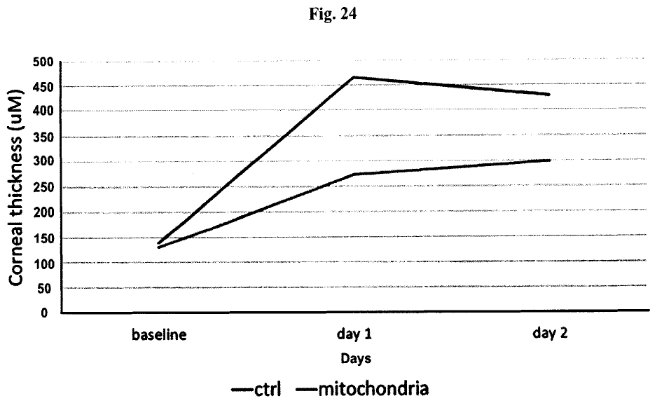

FIG. 24 is a graph showing the effect of exogenous mitochondria injection on the thickness of damaged cornea. Sprague-Dawley rats underwent transcorneal cryo-damage, and immediately after which mitochondria (10 million) or PBS (ctrl) were administered via intracameral injection. Corneal thickness was assessed by OCT 24h (day 1) and 48 h (day 2) after mitochondria administration;

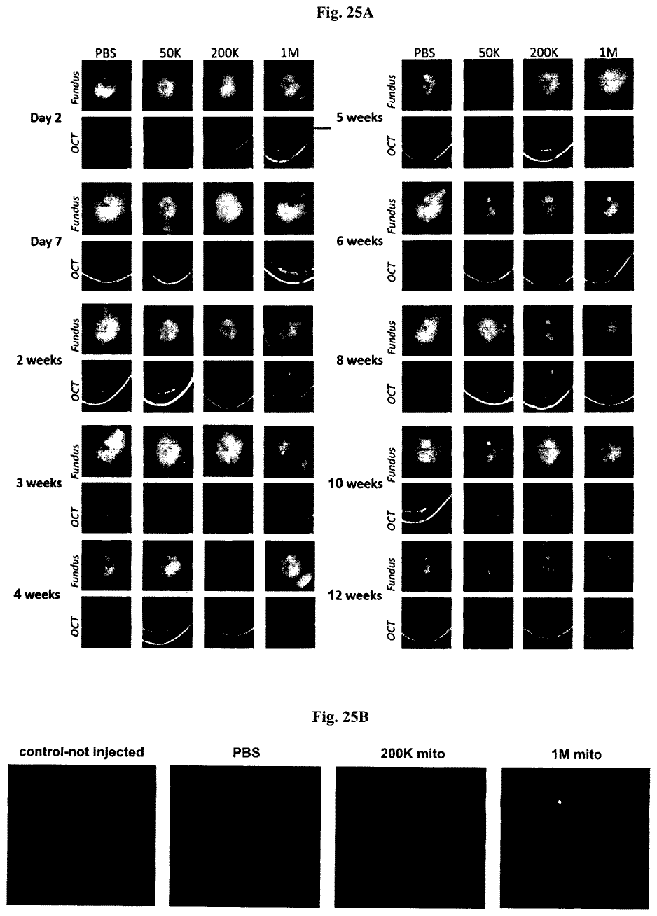

FIGS. 25A-25B present retinal anatomy in representative mice following bi-weekly intravitreal injections of PBS or mitochondria. The mitochondrial dose increased every two weeks, as indicated (K=thousand; M=million). FIG. 25A compiles OCT and fundus images arranged in 10 groups, each corresponding to a different time point from two days to 12 weeks. Each group includes four fundus images (upper row) and four OCT images (lower row) obtained after injection of PBS, 50 K, 200 K, or 1 M freshly isolated mitochondria. In the images collected two days after injection, arrows highlight an example of the injection site (1 M, rightmost OCT image) and excess mitochondria present in the vitreous 24 hours after injection (1 M, rightmost fundus image). FIG. 25B shows isolated retinae collected at the 12-week endpoint and immunostained with the RGC marker Tuj1; and

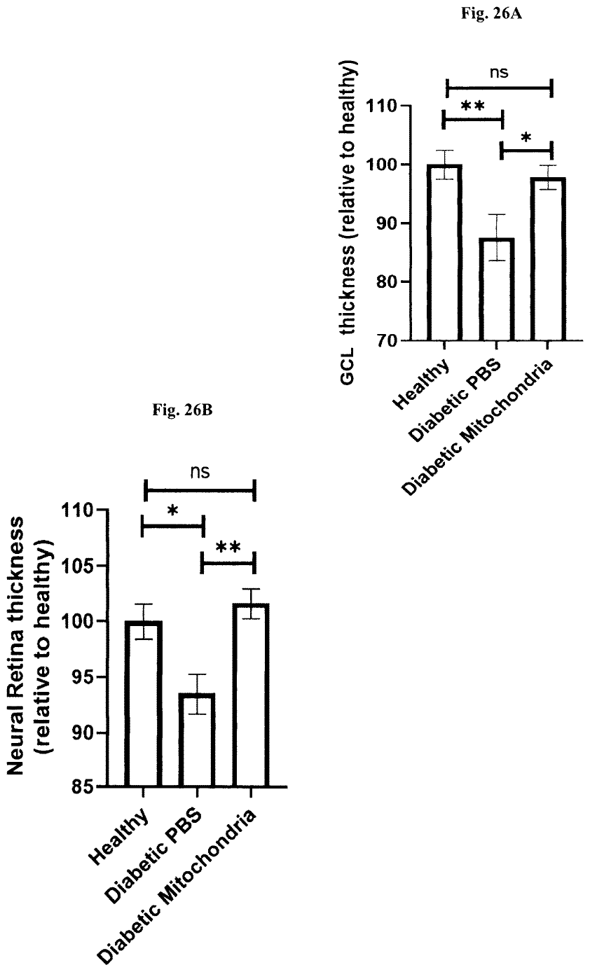

FIGS. 26A-26B are bar graphs illustrating the effects of mitochondrial transplantation on the retinas of diabetic mice. Diabetes was induced in C57B1/6 mice using streptozotocin, after which the animals received bi-weekly intravitreal injections of either isolated (exogeneous) mitochondria or phosphate-buffered saline (PBS). Healthy mice that did not receive streptozotocin served as controls. Retinal structure was evaluated by OCT at week 13. FIG. 26A depicts ganglion cell layer (GCL) thickness, and FIG. 26B shows neural retina thickness, each expressed relative to healthy controls for three groups: healthy mice, diabetic mice treated with PBS, and diabetic mice receiving exogenous mitochondria. Thickness measurements were obtained using ImageJ and reported as a percentage of the mean thickness in healthy mice. Statistical analysis was performed using ANOVA.

### DETAILED DESCRIPTION OF THE INVENTION

The present disclosure relates, at least in part, to ocular mitochondrial transplantation as means to treat diseases, disorders and conditions associated with, but not limited to, mitochondrial dysfunction in the eye.

The present disclosure is based on a discovery by the present inventors that fresh, isolated mitochondria injected into the vitreous cavity can be internalized by retinal ganglion cells (RGCs) and improve retinal function after ischemic insult in the retina. The present disclosure is based on a further discovery by the present inventors that damaged RGCs, e.g., following optic nerve crash, were significantly improved by transplanting freshly isolated mitochondria thereto. In yet a further discovery by the present investors, transplantation of mitochondria to the corneal epithelium layer significantly improved corneal healing after NaOH damage.

Disclosed herein are further discoveries by the present investors, wherein mitochondrial transplantation into damaged corneal endothelial cells dramatically improved their condition in terms of viability, migration ability and proliferation. Yet further discoveries presented herein relate to the successful treatment of degenerated retina in a diabetic retinopathy mice model.

Based on these discoveries, it has been envisaged by the present inventors that mitochondrial transplantation represents a promising therapeutic approach for a broad range of ocular diseases and conditions that could benefit from the introduction or replenishment of healthy, functional mitochondria.

Numerous retinal, corneal, and optic nerve pathologies are associated with impaired mitochondrial activity, oxidative stress, or age- and disease-related mitochondrial depletion. Introducing exogenous mitochondria, whether freshly isolated, preserved, or engineered, has the potential to restore cellular bioenergetics, enhance stress resilience, and support tissue repair. This is true also for ophthalmic conditions and pathologies which are not directly associated with, or caused by mitochondrial dysfunction.

Therapeutic delivery of exogeneous mitochondria can be tailored to the anatomical site of pathology. Depending on the disease, mitochondria may be administered directly into the vitreous cavity, anterior chamber, suprachoroidal space, subretinal space or applied to the corneal surface. In addition, systemic routes, such as intravenous injections, enable targeting of deeper ocular structures or diffuse mitochondrial deficits. This flexibility in delivery, combined with the central role of mitochondrial dysfunction in many blinding disorders, highlights the significant therapeutic potential of mitochondrial transplantation for ocular health.

Strategies of mitochondrial transplantation are known treatment paradigms attempted in treating mitochondrial dysfunction related, e.g., to injury and disease states such as central nervous system (CNS) diseases (e.g., Alzheimer's disease, Parkinson's disease), stroke, traumatic brain or spinal cord injury, acute lung injury, or cardiac ischemia. Already in 1982, successful mitochondrial transplantation was demonstrated in vitro when Clark and Shay found that simple co-incubation of isolated mitochondria with mammalian cells resulted in spontaneous internalization of the mitochondria by the cells (Clark and Shay, 1982, Somatic cell genetics, 8:15-21). Transplanted mitochondria were functional inside the recipient cells; they could increase oxygen consumption, ATP production and cell proliferation, and were able to replace dysfunctional mitochondria. In vivo mitochondrial transplantation is also known in the art. For example, injection of autologous mitochondria directly into ischemic rabbit hearts, immediately before reperfusion, resulted in increased mechanical heart function, smaller infarct size, higher ATP content and a significant reduction in troponin blood levels. Exogenous mitochondria have been taken-up by cardiomyocytes as early as 2 h after transplantation (McCully et al., 2009, American journal of physiology Heart and circulatory physiology, 296:H94-H105). Further in vivo studies are known, for example, in Gollihue et al., 2018, Neural Regeneration Research 13:194-197; Chen et al., 2016, J. Pineal Research 61:52-68; Kuo et al., 2017, Neurosurgery 80:475-488; Robicsek et al., 2018, Schizophrenia Bulletin 44:432-442; Shi et al., 2017, Mitochondrion 34:91-100; and Emani and McCully, 2018, Translational Pediatrics 7:169-175.

There are many benefits to replacing mitochondria in injury or disease states, including increasing energy production, increasing calcium buffering capacity, and replacing mitochondrial DNA.

“Mitochondrial transplantation”, herein also interchangeably used with terms such as “mitochondrial transfer”, “mitochondrial internalization” and “mitochondrial replacement”, refers to transplantation of mitochondria isolated from a healthy tissue into diseased and/or injured tissues. There are various techniques for transplanting exogenous mitochondria, and any scaffold that contains mitochondria, either synthetic or biological, may be employed in accordance with embodiments described herein.

Isolated mitochondria may be delivered to the eye via local and/or systemic delivery routes. For example, mitochondria may be directly injected or microinjected into the eye; topically applied; delivered via cell-mediated transfer, e.g., utilizing tunneling nanotubes; delivered via vesicle-mediated delivery, nanoparticle/polymer mediated delivery, and/or peptide-mediated delivery; and/or delivered within an amniotic membrane. Each of these results in mitochondria incorporation into host cells. Some of the various delivery techniques are schematically shown in FIG. 1A.

In cell-mediated transfer of mitochondria, a donor cell, whether implanted or endogenous, such as a bone marrow derived stromal cell (mBMSC), astrocyte or mesenchymal stem cell (MSC), secretes vesicles containing mitochondria, which are subsequently taken into recipient cells and tissues (e.g., brain neurons, lung tissue). Attachment of the mitochondria-donating cells (e.g., mBMSCs) via connexin pore gap junction formation appears necessary for this donation, with nanotubes formed between the host cells and mitochondria-containing vesicles (FIG. 1A, nanotube).

Microinjection is the use of a micropipette to inject the mitochondria at a microscopic or borderline macroscopic level, wherein the target is often a living cell but may also include intercellular space. Microinjection is usually conducted under a microscope (FIG. 1A, microinjection).

Mitochondria may also be transplanted using systemic delivery approaches (FIG. 1A, systemic) such as vascular perfusion of mitochondria, for example, through the coronary artery, or via vein injections. Like direct injection, systemic delivery techniques result in functional protection of injured tissues. Exogenous mitochondria systemically delivered may reach both targeted as well as untargeted tissues, whereby the uptake mechanism is most probably endocytosis. In diseases in which mitochondria dysfunction is more focal, direct transplantation of concentrated healthy mitochondria within that injured tissue is more applicable than systemic delivery.

Uptake of mitochondria-containing vesicles (FIG. 1A, vesicle-mediated) is probably via endocytosis, a phenomenon known as massive endocytosis. Once internalized, the mitochondria are released into the cytosol. The use of vesicles or liposomes reduces exposure of mitochondria to possible adverse effectors in the extracellular environment such as reactive oxygen and nitrogen species. A caveat to this technique is the difficulty in packaging high numbers of mitochondria within liposomes.

The amniotic membrane (amnion) is the innermost layer of the placenta and is composed of a thick basement membrane supported by a compact, avascular stromal matrix. Amniotic membrane transplantation has been widely used across surgical disciplines, functioning either as a biological graft or as a protective dressing. In ophthalmology, the amniotic membrane plays an important role in ocular surface reconstruction, serving as a graft for corneal melts, and as a therapeutic bandage to promote healing in cases of persistent epithelial defects, chemical injuries, or inflammatory ocular surface disorders.

Loading the amniotic membrane with exogenous mitochondria further enhances its therapeutic potential. The enriched membrane can deliver viable mitochondria directly to compromised ocular tissues, thereby augmenting cellular energy production, reducing oxidative stress, and accelerating regenerative processes. In this manner, mitochondria-loaded amniotic membrane can significantly improve healing outcomes, particularly for the corneal epithelium and other metabolically stressed ocular surface tissues.

Embodiments described herein pertain to transplantation, by either route, of freshly isolated mitochondria which may be non-autologous, e.g., mitochondria isolated from cell culture, or autologous.

In accordance with embodiments described herein, before transplantation, the functionality and integrity of transplanted mitochondria is monitored, for example, by electron microscopy for assessing morphology and purity, by measuring mitochondrial respiration and energy production, by assessing membrane potential, for example, by using fluorescence membrane potential dyes and/or by measuring calcium buffering.

### Methods of Treatment

Mitochondrial dysfunction has a critical role in many ocular diseases. In addition, the eye is highly susceptible to ischemia. Yet, mitochondrial transplantation in the eye has not been attempted. The RGC layer is the first cell layer of the retina having direct contact with the vitreous. The present inventors envisaged that mitochondria injected into the vitreous cavity may be internalized by the RGCs. Indeed, the present inventors have successively demonstrated, for the first time, mitochondria internalization into the retina, and more specifically into RGCs, as described in Example 5 herein. Moreover, as described in Examples 6 and 7 herein, damaged retinal function after ischemic insult has been improved by transfer of mitochondria into RGCs. Furthermore, Example 18 herein demonstrates that mitochondria transplantation was effective in alleviating and improving retinal degeneration in a moder of diabetic retinopathy.

The present inventors have further demonstrated that mitochondrial transplantation to the injured corneal epithelium markedly enhanced both the rate and completeness of corneal wound healing (see Examples 9 and 10 herein). In addition, transplantation of mitochondria to corneal endothelial cells modulated oxidative stress-associated cellular reorganization, increased cell culture density, and improved cell migration capacity (see Examples 11, 12, and 14 herein). Together, these effects facilitated the repair of corneal damage, exemplified in vivo as reduction of corneal thickness post injury (Example 17 herein).

In one aspect, the present disclosure relates to a method for treatment of mitochondrial dysfunction, mitochondrial destruction or depletion in the eye of a subject, the method comprising administering to the eye of the subject a therapeutically effective amount of isolated mitochondria, thereby treating mitochondrial dysfunction, mitochondrial destruction or depletion.

Embodiments of the present disclosure utilize the disclosed method for treatment of various pathologies of the eye related to, or associated with, mitochondrial dysfunction or impairment, whereby, according to at least some of these embodiments, a therapeutically effective amount of allogeneic, syngeneic, xenogeneic and/or autologous exogenous mitochondria is being transferred to the eye by any of the means and routes described herein.

“Allogeneic”, as used herein, refers to cells, tissues, or biological materials derived from a donor who is genetically distinct from, but of the same species as, the recipient. Allogeneic materials possess genetic differences sufficient to elicit an immune response when introduced into the recipient, unless otherwise modified or immunologically matched.

The terms “allogeneic mitochondria transfer” and “allogeneic mitochondria transplant”, as use herein, are interchangeable and refer to mitochondria taken from one individual and transplanted into another individual of the same species but with a different genotype. For example, an allogeneic mitochondria transplant is mitochondria transplant donated from one person to another person who is not an identical twin, but may be, for example, a living related, or living unrelated acceptor. For example, allogeneic mitochondria may be extracted from a tissue (e.g., placenta) of one person and transferred to tissues of another person of a different genotype. Allogeneic mitochondria transplant also includes transplanted mitochondria extracted from a cell culture.

The term “syngeneic”, as used herein, means genetically identical, or sufficiently identical and immunologically compatible as to allow for transplantation. For example, syngeneic mitochondria or syngeneic mitochondria transplant may be obtained from an identical twin.

The terms “autologous mitochondria transfer” and “autologous mitochondria transplant”, as used herein, are interchangeable and refer to mitochondria taken from the same person, for example from a healthy tissue or organ of the a person, and transferred to/transplanted in a diseased or injured tissue or organ of that same person.

The term “xenogeneic” as used herein refers to cells, tissues, organs, or biological materials derived from an individual of a different species than the recipient. Xenogeneic materials typically contain substantial genetic and antigenic differences and therefore have a high potential to induce strong immune responses when introduced into another species.

The terms “xenogeneic mitochondria transfer” and “xenogeneic mitochondria transplant”, as used herein, are interchangeable and refer to mitochondria derived from, or originating in, a different species. For example, a xenogeneic mitochondria transplant is mitochondria transplant donated from mouse or rabbit to a human subject.

The term “exogenous mitochondria”, as used herein, refers to allogeneic, autologous, xenogeneic or syngeneic mitochondria that have been made to enter into, or have been transferred to, a cell or tissue by any of the natural or artificial known means for internalization. Exogenous mitochondria are to be distinguished from endogenous mitochondria, which are the naturally occurring mitochondria in the cells. Exogenous mitochondria may be isolated from their natural cellular environment/components and be purified or at least partly purified.

Mitochondria consist of four structural components: the outer membrane, inner membrane, intermembrane space, and matrix. They carry out multiple metabolic functions, including pyruvate oxidation, the Krebs cycle, and amino-acid, fatty-acid, and steroid metabolism. But their primary role is ATP production through the electron transport chain and oxidative phosphorylation.

Cellular ATP generation depends on the mitochondrion's ability to use metabolic substrates to generate nicotinamide adenine dinucleotide (NADH), transfer electrons from NADH to the respiratory chain, and ultimately reduce molecular oxygen. Electron transfer drives proton pumping from the matrix to the intermembrane space, establishing a proton gradient (Δp) and membrane potential (Δψm) across the inner membrane. ATP synthase then uses the energy released by proton flow back into the matrix to convert ADP to ATP.

Mitochondrial dysfunction can arise from an insufficient number of mitochondria or impaired electron-transport and ATP-synthesis machinery. It is a hallmark of aging, certain injuries (e.g., ocular injury), and various chronic diseases. Mitochondrial dysfunction may also be congenital, resulting from mutations or other alterations in nuclear or mitochondrial DNA.

Functionally, mitochondrial dysfunction is defined by reduced efficiency of the electron transport chain and diminished synthesis of high-energy molecules such as ATP. At the molecular level, impaired mitochondrial performance may result from: (1) failure to maintain the electrical and chemical potential across the inner mitochondrial membrane; (2) defects in electron-transport chain components; or (3) decreased transport of critical metabolites into the mitochondria.

Electron transport inherently generates reactive oxygen species (ROS) and related reactive nitrogen species (RNS) as by-products of oxidative phosphorylation. Because mitochondria are the principal source of ROS/RNS, dysfunction can elevate their production, leading to oxidative damage to lipids, proteins, and DNA. Impaired electron transport may also promote protein uncoupling, allowing uncontrolled proton leak across the inner mitochondrial membrane and partial dissipation of the electrochemical gradient. This proton leak reduces ATP output and contributes to oxidative damage of mitochondrial lipids, particularly cardiolipin, a ROS/RNS-sensitive phospholipid that stabilizes electron-transport chain complexes. Oxidation of cardiolipin disrupts these complexes and further compromises electron-transport function.

The number and functional status of mitochondria within a cell can be modified, including enhanced, through naturally occurring or artificially induced intracellular processes. These include: (1) fusion of partially impaired mitochondria, allowing mixing of intact components to restore function; (2) biogenesis of new mitochondria through fission; and (3) selective removal and degradation of dysfunctional mitochondria via mitophagy. These processes are regulated by cellular mechanisms that detect mitochondrial damage, such as loss of membrane potential or activation of specific transcriptional pathways.

In addition, or as an alternative, mitochondrial number and function may be modified through extracellular interventions, including mitochondrial transplantation as described herein.

The terms “mitochondrial disease”, “mitochondrial disorder” and “mitochondrial condition”, as used herein, are interchangeable and in the context of some embodiments described herein, refer to a disease, disorder or condition directly or indirectly caused by, exacerbate by, worsen by, a sequelae of, or deteriorated due to, mitochondrial dysfunction as defined herein.

Mitochondrial diseases are categorized as either primary or secondary to indicate their underlying etiology. Primary mitochondrial diseases result from direct impairment of mitochondrial functions due to modifications (e.g., mutations) of genes located in either nuclear DNA (nDNA) or mitochondrial DNA (mtDNA), which is more prone to mutagenesis (~10-fold greater than in nDNA). All but 13 of the more than 1,000 proteins necessary to mitochondrial constitution and function are encoded by nuclear genes. Secondary mitochondrial dysfunction results either from environmental factors and/or other genetic disorders which affect mitochondrial function.

Nuclear gene defects causing primary mitochondrial diseases may follow autosomal recessive or dominant inheritance. In contrast, mitochondrial DNA (mtDNA) is inherited exclusively from the mother because all mitochondria in the zygote originate from the ovum. Thus, a mother carrying an mtDNA mutation transmits it to all her children, but only her daughters can pass it to the next generation. The proportion of mutant mtDNA each child receives can vary widely, producing substantial clinical variability among siblings.

Each cell contains thousands of mtDNA copies, and pathogenic mutations typically affect only a subset of them. Cells and tissues therefore contain a mixture of normal and mutant genomes, a condition known as heteroplasmy. Heteroplasmy may also occur within individual mitochondria. Most pathogenic mtDNA mutations are point mutations, and clinical diseases appear only when the proportion of mutated mtDNA exceeds a critical threshold. This threshold is lowest in tissues highly reliant on oxidative metabolism such as the brain, heart, skeletal muscle, retina, renal tubules, and endocrine glands, making them particularly vulnerable.

Clinical syndromes may arise from multiple different mtDNA mutations, and a single mutation can produce diverse phenotypes depending on heteroplasmy levels. Consequently, genotype-phenotype correlations are often unpredictable. For example, mutations commonly linked to MELAS (mitochondrial encephalomyopathy, lactic acidosis, and stroke-like episodes) can produce other syndromes, while mutations in different genes can lead to the same clinical presentation, including MELAS.

In some embodiments, the eye pathology associated with mitochondrial dysfunction or impairment, which is treatable by a disclosed method, is an ocular manifestation of a systemic disease or condition in a subject. In accordance with these embodiments, a therapeutically effective amount of exogenous, isolated mitochondria is administered to the subject for treating the ocular manifestation of a systemic disease or condition.

The term “ocular manifestation of a systemic disease”, as referred to herein, is an eye condition that directly or indirectly results from a primary non-ocular disease process or injury in another part of the body. There are many diseases and conditions known to cause ocular or visual changes, such as, but not limited to, congenital, vascular, neoplastic, autoimmune, idiopathic, infectious, metabolic/endocrine, and/or hypertension diseases. Non-limiting examples of conditions which may have ocular manifestation include drugs usage, trauma and/or exposure to toxins.

Any systemic disease or injury manifested in the eye as impaired oxygen supply, ischemia (e.g., ischemic infarction of the inner retina), hemorrhages, occlusion of, or changes in, blood vessels (e.g., retinal embolization, central retinal vein occlusion (CRVO)), that is associated with mitochondrial dysfunction, mitochondrial destruction and/or mitochondrial depletion in the eye, for example, in the retina, can be treated by a disclosed method.

Diabetes, for example, has ocular manifestations such as diabetic retinopathy and macular edema are the leading cause of blindness in diabetic patients. Other diseases such as acquired immunodeficiency syndrome (AIDS), and hypertension are commonly found to have associated ocular symptoms. Systemic infection may also be manifested by ocular signs and symptoms. Ocular manifestations often prevail in numerous congenital syndromes, including Down syndrome, Marfan syndrome, myotonic dystrophy, tuberous sclerosis, metabolic disorders involving lysosomal storage and carbohydrate metabolism, and neurofibromatosis.

In some embodiment, the eye pathology associated with mitochondrial dysfunction or impairment which is treatable by a disclosed method is an ophthalmic disease, disorder or condition. In accordance with these embodiments, a therapeutically effective amount of exogenous, isolated mitochondria is administered to the subject to thereby treat the ophthalmic disease, disorder or condition.

As used herein, the phrases “ophthalmic disease, disorder or condition associated with mitochondrial dysfunction” and “ophthalmic disease, disorder or condition associated with a mitochondrial disease or disorder” are interchangeable and refer to an ophthalmic pathology affecting the normal state and function of the eye, which is directly or indirectly caused by, exacerbate by, or a sequelae of, a mitochondrial disease as defined herein. The eye is one of the most affected organs in mitochondrial diseases, with devastating effects that may variably involve the extraocular eye muscles, levator palpebrae superioris muscle, lens, retina, and/or optic nerve. The etiology of many common ocular disorders is increasingly recognized to involve either an accumulation of mitochondrial DNA mutations and/or secondary mitochondrial damage.

Many primary mitochondrial diseases have ophthalmologic involvement, commonly with significant phenotypic overlap that may prohibit ready distinction of a specific genetic etiology based on clinical parameters alone. To further complicate matters, mutations in a given mitochondrial gene may be associated with a spectrum of different clinical phenotypes. The four most common neruophthalmic abnormalities seen in mitochondrial disorders are bilateral optic neuropathy, ophthalmoplegia and ptosis, pigmentary retinopathy and retro chiasmal visual loss. Non-limiting examples of utilizing a contemplated method disclosed herein for the treatment of ophthalmic diseases, disorders or conditions associated with a primary mitochondrial disease or disorder are described below. Treatment, in accordance with these embodiments, comprises transplantation of exogenous isolated mitochondria directly to the eye by any of the routes described herein, and/or by any applicable systemic administration route.

In some embodiments, the ophthalmic disease is dominant optic atrophy (DOA), a genetic disease that primarily affects the RGC and nerve fiber layer of the retina. Visual acuity typically decreases over the first two decades of life to a mean of from 20/80 to 20/120. Thinning of the neuroretinal rim appears to be a universal finding in DOA. Mutations in the nuclear-encoded dynamin-like GTPase nuclear gene OPA1, which is involved in mitochondrial fusion, are responsible for the majority of DOA cases. OPA1 mutations can cause mitochondrial disease even in the absence of optic atrophy (i.e., partial or complete wasting away of parts of the eye such as the optic nerve). In acute states, in accordance with a contemplated method, DOA can be treated, for example, by one or more injections of isolated mitochondria. Repeated injections of isolated mitochondria may be the treatment modality of choice for chronic states.

In some embodiments, the ophthalmic disease is Leber hereditary optic neuropathy (LHON), a bilateral optic neuropathy characterized by acute and painless central vision loss of both eyes in a sequential fashion over a period of days to months. The onset of visual loss typically occurs between the ages of 15 and 35 years. Visual acuities in LHON patients at the point of maximum visual loss range from no light perception to 20/20 with most patients deteriorating to acuities worse than 20/200. Color vision is severely affected, often early in the course, but rarely before, considerable acuity loss. The pathogenesis of LHON involves initial thickening of the retinal nerve fiber layers with disc pseudoedema and RGC loss within the optic nerve. LHON is a maternally inherited ophthalmologic disorder linked to at least three point-mutations in mitochondrial DNA, mainly in genes coding for proteins involved in respiratory chain complex I activity. Currently, no cure for LHON exists, although various approaches are attempted in improving visual outcome, including gene therapy approaches.

In some embodiments, the ophthalmic disease or disorder is chronic progressive external ophthalmoplegia (CPEO). “Ophthalmoplegia”, as used herein, refers to the paralysis or weakness of the eye muscles. It is a complex disorder that impairs bilateral extraocular muscle mobility and can affect one or more of the six muscles that hold the eye in place and control its movement. CPEO is associated with ptosis but rarely with diplopia (double vision). Visual acuity is typically spared, although some patients may develop optic atrophy or retinal involvement. The disease is most commonly caused by a single mtDNA deletion mutation. The disease may be “sporadic” in that the mtDNA deletion occurs de novo in the affected individual and is unlikely to be transmitted to an affected individual's progeny. Other structural rearrangements or point mutations in mtDNA may also result in CPEO that can be transmitted in a maternal fashion. In addition, multiple mtDNA deletions or duplications can be the cause of the disease and result from mutations in any of a number of nuclear genes that are involved in mitochondrial DNA maintenance.

Chronic progressive external ophthalmoplegia is also a very common ocular manifestation of mitochondrial myopathies associated with other neurological or systemic abnormalities, such as bulbar and limb myopathies, deafness, ataxia, spasticity, peripheral neuropathy, gastrointestinal myopathy and the like. Associated ocular features include optic atrophy, pigmentary retinopathy, corneal changes, cataracts and Kearns-Sayre syndrome (KSS). The term “myopathy” means a muscle disease, in which the muscle fibers do not function properly. This results in muscular weakness and sometimes also in muscle stiffness and spasm. The term “mitochondrial myopathy”, as used herein, refers to forms of mitochondrial disease that cause prominent muscle problems.

In accordance with a contemplated method, treatment of CPEO may comprise transplantation of isolated mitochondria directly to the eye, e.g., to an extraocular muscle, and/or by systemic administration of isolated mitochondria.

In some embodiments, the ophthalmic disease or disorder treatable by a contemplated method is pigmentary retinopathy. Retinopathy is any damage (e.g., persistent or acute) to the retina, which may cause vision impairment. Retinopathy often refers to retinal vascular disease or damage to the retina caused by abnormal blood flow. Frequently, retinopathy is an ocular manifestation of a systemic disease such as diabetes or hypertension.

Pigmentary retinopathy (PR) refers to a group of inherited, degenerative disorders of the retina, characterized by progressive photoreceptor damage, leading to atrophy and cell death of the photoreceptors and adjacent layers of the retina. Pigmentary retinopathy primarily affects the rods and consequently the cones, causing blindness in advanced cases when central retina is involved. The initial symptoms of the disease include nyctalopia (night blindness), peripheral visual field constriction, and sometimes loss of the central visual acuity or visual field. Pigmentary retinopathy may be seen in the primary mtDNA disease neurogenic weakness, ataxia, and retinitis pigmentosa (NARP), which results from a point mutation in mitochondrial DNA.

Pigmentary retinopathy is the final common outcome of many retinal and chorioretinal disorders and often a common manifestation of numerous metabolic and neurodegenerative diseases. Pigmentary retinopathy can also occur in a range of other mtDNA cytopathies including Leigh syndrome (degenerative disorder involving the basal ganglia and brainstem), MELAS, myoclonic epilepsy and ragged red fibers (MERRF), LHON, KSS and mitochondrial myopathy.

In accordance with a contemplated method, treatment of pigmentary retinopathy comprises transplantation of exogenous isolated mitochondria directly to the eye, e.g., to the vitreous and/or by systemic administration of isolated mitochondria.

In some embodiments, the ophthalmic disease or disorder treatable by a contemplated method is Kearns-Sayre syndrome (KSS). Kearns-Sayre syndrome is a progressive external ophthalmoplegia (PEO) and pigmentary retinopathy with onset before 20 years of age. This syndrome is associated with mtDNA rearrangements (large deletions or duplications). It is always heterogenous and the concentration of the rearranged mtDNA in different tissues determines the age and manner of presentation, not all conform to the syndromes. Most patients with Kearns-Sayre syndrome develop a “salt and pepper” fundus. Visual acuity (acuteness) tends not to be severely impaired unless there is optic atrophy (degeneration). Other patients have generalized loss of the retinal pigment epithelium.

In accordance with a contemplated method, treatment of KSS comprises transplantation of exogeneous isolated mitochondria directly to the eye, e.g., to an extraocular muscle, the vitreous and/or by systemic administration of isolated mitochondria.

In some embodiments, the ophthalmic disease or disorder is corneal clouding, a rare manifestation of KSS with corneal clouding associated with either congenital glaucoma or corneal dystrophy (degeneration). Structural changes in endothelium and Descemet's membrane have been reported with corneal edema that may be due to reduced pump action of corneal mitochondria. Treatment of corneal clouding, in accordance with a contemplated method, comprises transplantation of isolated mitochondria directly to the eye, e.g., to the cornea and/or to the anterior chamber. Alternatively, or additionally, mitochondrial transfer to the eye is via topical administration of eye drops.

Ophthalmologic diseases which are not considered as originating from primary mitochondrial diseases, are increasingly recognized to result, at least in part, from secondary, i.e., non-genetic, mitochondrial diseases or disorders (also referred to herein as “secondary mitochondrial dysfunction”), exemplified in a non-limiting manner by increased oxidative stress, and/or increased apoptosis, which are mediated by mitochondria. For example, oxidative damage that results over time from mtDNA instability leads to cumulative mitochondrial damage, which is recognized to be an important pathogenic factor in age-related ophthalmologic disorders such as diabetic retinopathy, age-related macular degeneration, and glaucoma.

A contemplated method is applicable for the treatment of ophthalmic diseases, disorders or conditions associated with a secondary mitochondrial disease or disorder such as, but not limited to, diabetic retinopathy.

In a further example, the ophthalmic disease, disorder or condition treatable with a contemplated method is glaucoma, the second-leading cause of blindness worldwide. It is an optic neuropathy that manifests with optic nerve cupping and atrophy similar to what is observed in primary mitochondrial optic neuropathies. The optic nerve is packed with mitochondria, making it particularly susceptible to impairment of mitochondrial respiratory capacity that can selectively damage RGC. Although mitochondrial function may be impaired by mutations in either nuclear or mitochondrial DNA in glaucoma, mechanical stress or chronic hypoperfusion caused by increased intraocular pressure, toxic xenobiotics, or even light-induced oxidative stress, play a major role in causing secondary mitochondrial dysfunction encountered in glaucoma patients.

Further classic ophthalmologic diseases characterized by secondary mitochondrial dysfunction are exemplified by maternally inherited diabetes and deafness, age-related macular degeneration (AMD), proliferative vitreo-retinopathy (PVR), and Fuchs dystrophy (in corneal endothelium). Any of these diseases are treatable by mitochondrial transplantation in accordance with a contemplated method disclosed herein.

Secondary mitochondrial dysfunction resulting directly or indirectly from external environmental factors such as local injury or trauma in the eye or exposure to toxic materials may lead to ophthalmic conditions or disorders. Such conditions may be acute or mild and are treatable by mitochondrial transplantation as described herein.

In some embodiments, the secondary mitochondrial dysfunction, which may be alleviated, relieved or cured is ischemia. Ischemia is broadly defined as the loss of blood supply to a biological tissue, resulting in energy depletion and often even cell death, both of which are mediated by intermediate factors such as the release of excess excitatory amino acids, free-radical formation, and inflammation. Retinal ischemia is a common cause of visual impairment and blindness and is a characteristic feature of various clinical retinal disorders such as ischemic optic neuropathies, obstructive arterial and venous retinopathies, carotid occlusive disorders, retinopathy of prematurity, chronic diabetic retinopathy and glaucoma. A hallmark of these pathologies is the death of retinal ganglion cells.

Retinal ischemia occurs when the retinal circulation is insufficient to meet metabolic demands. It can be caused by general or, more commonly, by local circulatory failure. The metabolic demands of the retina are the highest of any tissue within the body, thus, maintaining a consistently high blood supply is essential.

There are many pathogenic mechanisms which contribute to the cell death cascades experienced during ischemia, such as energy failure, elevation of intracellular calcium, generation of free radicals, blood-retinal barrier disruption, inflammation and apoptosis. At least some of these mechanisms, directly or indirectly, affect mitochondrial function, and mitochondrial dysfunction ultimately leads to cell death.

Currently, no treatment is available for rescuing dying cells in acute ischemic insults, except for opening the occult artery that causes ischemia (if possible). Hence, a treatment that targets the compromised cells and improves their survival, and thus the patient prognosis, is an unmet need.

Embodiments described herein pertain to treatment of various forms of ischemic optic neuropathies, also interchangeably referred to herein as “ocular ischemic insults”, by means of transplantation of intact, vital, functioning exogenous mitochondria to diseased parts of the eye. As shown in the Examples described herein, freshly isolated mitochondria injected in vivo to ischemic tissue, were internalized by compromised cells and protected them from death. It is assumed that a new reservoir of healthy mitochondria decreases ROS production within ischemic tissues, provides a new pool of intact, exogenous mitochondrial-DNA and increases energy production and calcium buffering capacity. For example, a contemplated method is useful in treatment of non-arteritic anterior ischemic optic neuropathy (NAION), central retinal artery occlusion (CRAO), or acute angle closure glaucoma (AACG).

Some embodiments disclosed herein pertain to the treatment of retinal degeneration.

The term “retinal degeneration,” as used herein, refers to a progressive deterioration of the retina, manifesting as gradual loss of retinal cells: photoreceptors, RPE, ganglion cells, as well as other retinal cells such as Muller cells or vascular endothelium, and consequent decline of visual function. Retinal degeneration is a defining feature of numerous inherited, metabolic, vascular, inflammatory, mitochondrial, toxic, drug-induced and/or age-related insults, diseases or disorders. It encompasses diseases primarily driven by mitochondrial dysfunction (e.g., LHON, KSS, NARP, as well as conditions where mitochondria are not the primary cause (e.g., diabetic retinal neurodegeneration, glaucoma, AMD, retinitis pigmentosa due to non-mitochondrial gene defects, autoimmune retinopathies, vascular occlusions). Across this spectrum, progressive loss or dysfunction of the retinal cells constitutes the central pathological hallmark.

Retinal degeneration can arise from multiple molecular pathways that can be broadly categorized into mechanisms involving mitochondrial dysfunction and mechanisms not directly related to mitochondrial pathology. Because these diverse pathways converge on similar cellular outcomes, such as photoreceptor loss, RPE atrophy, inner retinal neurodegeneration, and structural disorganization, the term “retinal degeneration” encompasses a wide range of phenotypes and etiologies. Mitochondrial-related and mitochondrial non-related retinal degeneration processes can be prevented, alleviated, treated or mitigated by transplantation of exogeneous mitochondria in accordance with the methods disclosed herein.

Mitochondrial dysfunction represents a major and often early driver of retinal degeneration. Primary mitochondrial defects, such as mutations in mtDNA or nuclear genes encoding mitochondrial proteins, can impair oxidative phosphorylation, ATP production, and mitochondrial homeostasis, leading to progressive retinal cell loss. Inherited mitochondrial diseases including Leber hereditary optic neuropathy (LHON), neuropathy, ataxia, retinitis pigmentosa (NARP), Kearns-Sayre syndrome (KSS), and maternally inherited diabetes and deafness (MIDD) exemplify conditions in which defective mitochondrial function results in degeneration of photoreceptors, RPE cells and/or retinal ganglion cells. Beyond inherited defects, age-related or stress-induced mitochondrial damage is common in the retina, particularly in the metabolically demanding RPE and neural retina.

Chronic oxidative stress, metabolic imbalance, and environmental insults render retinal mitochondria especially vulnerable. Dysfunctional mitochondria may generate excessive reactive oxygen species, fail to maintain membrane potential, or undergo inadequate mitophagy, producing a toxic intracellular environment that triggers inflammation, activation of apoptotic pathways, and eventual retinal cell death. In many diseases, including age-related macular degeneration (AMD), mitochondrial impairment is thought to precede measurable cell loss and may drive early disease progression. Accordingly, mitochondrial dysfunction acts as a central or initiating event in a substantial subset of retinal degenerative conditions.

Retinal degeneration also arises from numerous mechanisms that do not primarily involve mitochondrial dysfunction. Many inherited retinal disorders and dystrophies such as retinitis pigmentosa, cone-rod dystrophy (characterized by primary cone photoreceptor degeneration with subsequent rod involvement), Leber congenital amaurosis, and various inherited maculopathies, are caused by mutations that disrupt photoreceptor structure, phototransduction, RPE maintenance, or retinal development. These non-mitochondrial genetic defects often produce characteristic patterns of photoreceptor degeneration and visual decline. Age-related degenerative processes likewise contribute to retinal deterioration, as in AMD, where genetic susceptibility, chronic inflammation, environmental stressors, and dysregulated lipid or complement pathways act independently of direct mitochondrial injury.

Vascular abnormalities represent another major source of retinal degeneration: ischemia, vascular insufficiency, or pathological neovascularization can deprive retinal tissues of oxygen and nutrients (collectively referred to as “nutrient deficiencies”), resulting in progressive cell loss. This mechanism is particularly evident in “wet” AMD, retinal vein or artery occlusions, and other ischemic retinopathies.

Additionally, chronic inflammation and immune-mediated injury, such as in autoimmune retinopathy, chronic uveitis, or complement-driven macular diseases, can cause cumulative retinal damage leading to degeneration. Infectious, traumatic, and toxic insults may further contribute to retinal atrophy and neuronal loss through pathways that do not primarily involve mitochondrial dysfunction.

Glaucoma, primarily an optic neuropathy wherein the primary site of damage is the optic nerve, involves progressive loss of retinal ganglion cells (RGCs) and their axons, leading to characteristic visual field loss. Because the retina is affected secondarily, via RGC loss, glaucoma is also referred to herein as a secondary retinal degeneration disease.

Although retinal degeneration may arise from many mechanisms, both mitochondrial and non-mitochondrial processes frequently converge on similar degenerative outcomes. Notably, even conditions traditionally considered mitochondria-independent can benefit from therapeutic approaches targeting mitochondrial function, including transplantation of exogenous mitochondria as disclosed herein. Because of the retina's exceptional energetic demands and the central role of mitochondria in maintaining cellular health, enhancing mitochondrial integrity prevents, mitigated, or slowed degeneration across a range of etiologies.

In some embodiments, the disclosed method is applied for prevention or treatment of diabetic retinopathy (also referred to herein as “diabetic retinal neurodegeneration”).

Diabetic retinopathy (DR) is a leading cause of adult blindness worldwide. About one-third of individuals with diabetes develop DR, of which roughly one-third progress to sight-threatening stages. DR involves both vascular dysfunction and progressive retinal neurodegeneration. Although anti-VEGF therapies are standard care, 15-20% of patients show poor response, underscoring the need for novel treatment strategies.

Oxidative stress is a central driver of DR pathogenesis. Chronic hyperglycemia promotes the buildup of glucose-derived oxidative products that directly injure the mitochondria. This is particularly harmful in the retina, where energy demands are exceptionally high; for instance, a single retinal ganglion cell (RGC) requires ~4.68×10⁸ ATP molecules per second to sustain visual signaling. In diabetes, mitochondrial damage disrupts the electron transport chain, lowers membrane potential, and impairs oxidative phosphorylation, leading to critical ATP deficits.

Mitochondrial dysfunction also activates neurodegenerative pathways. Damaged mitochondria trigger intrinsic apoptosis, contributing to early and significant RGC loss in DR. Accumulation of dysfunctional mitochondria further elevates oxidative stress and inflammation through defective mitophagy and release of mitochondrial DNA, which activates innate immune responses. In addition, impaired mitochondrial function increases VEGF production, worsening pathological vascular changes and accelerating retinal injury.

The therapeutic approach disclosed herein (see Example 18), involving intravitreal delivery of isolated mitochondria, is shown to be safe and capable of counteracting diabetes-induced retinal damage, demonstrating efficacy in a complex and chronic in-vivo disease setting. Moreover, the disclosed method proved safe in multiple injections, every couple of weeks for a few months.

The cornea is the transparent, outermost layer of the eye that plays a crucial role in focusing light onto the retina. The cornea has five layers: (1) epithelium: the outermost, protective layer of the cornea; (2) Bowman's membrane: a strong second protective layer; (3) stroma: the thickest layer of the cornea. It is made up of water, collagen fibers and other connective tissue, which strengthen the cornea and make it flexible and clear; (4) Descemet's membrane: a thin, strong inner layer that is also protective; and (5) endothelium: the innermost layer made up of cells that pump excess water out of the cornea.

Damage to the cornea can cause significant vision loss and is a major cause of blindness worldwide. For example, chemical injuries to the cornea can result in a range of complications such as corneal opacity, ulceration, and perforation. The severity of the injury depends on several factors such as the type, concentration, and duration of exposure to the injuring agent.

Embodiments disclosed herein relate to the prevention, amelioration, treatment or mitigation of corneal pathologies such as local injury, trauma, dystrophy or disease, by virtue of curing mitochondrial dysfunction in the cornea.

In some embodiments, damage to the cornea is caused by exposure to chemicals.

Chemical injuries to the eye are serious and potentially vision-threatening conditions that can result from exposure to a variety of substances such as acids, alkalis, and other corrosive agents. Among these, alkali injuries are more common and often cause more severe damage than acid injuries. Sodium hydroxide (NaOH) and other corrosive alkali substances can cause significant damage to the eye, particularly damaging to the cornea, because they can penetrate the tissue more deeply and cause liquefactive necrosis, leading to rapid and extensive tissue damage. Corneal healing after chemical injury is often slow and incomplete and can result in long-term visual impairment or blindness.

In some embodiments, mitochondria transplantation is applied to damaged epithelium of the cornea in a subject in need thereof.

As used herein, the terms “damaged cornea epithelium” and “corneal wound” are interchangeable.

In some embodiments, damage to the cornea epithelium is caused by exposure to a chemical.

Injury to the corneal epithelial surface can result in epithelial defects. Healing occurs in three distinct phases characterized by epithelial cell migration, proliferation, and differentiation. Trauma to the corneal epithelium induces migration of the remaining epithelial cells adjacent to the injury site toward the defective area. Changes in cell-cell and cell-matrix (fibronectin-integrin system) interactions and modulation of the extracellular matrix by newly expressed proteolytic enzymes play important roles.

Current treatments of alkali damages to the corneal epithelium include irrigation with copious amounts of water or saline, administration of topical medications such as antibiotics and corticosteroids and, in some cases, surgical interventions such as amniotic membrane transplantation or corneal transplantation. However, despite these treatments, the rate and completeness of corneal healing after chemical injury remains limited, necessitating new and effective approaches to promote faster and more complete corneal healing after chemical injury.

The present inventors have shown that transplantation of mitochondria to the corneal epithelium layer significantly improved corneal healing after NaOH damage. In particular, the corneal epithelial wound closure rate observed was much faster in the group that received mitochondria transplantation compared to the control group without mitochondrial transplantation. In fact, as shown in Example 9 herein, the corneal wound treated with mitochondria transplantation closed completely after only 4 days, whereas in untreated eye, wound healing was no more than about 45%. H&E staining of corneas from control mice demonstrated broad damage including thinning and cell loss of the epithelial layer and thickening and disorganization of the stroma. However, corneas from mice transplanted with mitochondria had a multi-layered epithelium, and normal stroma thickness and organization, supporting a phenotype of repaired epithelium and proper corneal hydration. These findings suggest that mitochondria transplantation to the corneal epithelium layer can significantly improve the rate and completeness of corneal wound healing after chemical damage.

It is further demonstrated herein that transplantation of healthy mitochondria can lead to faster cell proliferation and improved wound healing in primary human corneal epithelial cells isolated from human corneas and cultured. As demonstrated in Example 10 herein, mitochondria transplantation induced proliferation of human corneal epithelial cells by approximately 15-20%. The wound healing effect of mitochondria transplantation is therefore likely a combination of enhanced proliferation, migration and potentially cell survival mechanisms.

Thus, it has been successfully demonstrated by the present inventors, and for the first time, that transplanted mitochondria are highly effective in corneal wound healing. Furthermore, this approach demonstrated minimal adverse effects, making it a potentially safe and effective treatment for ocular injuries such as retinal and corneal injuries.

Other corneal epithelial defects treatable by a disclosed method can occur by a variety of other causes, such as, but not limited to: dry eye and decreased tear production (due to side effects of topical or systemic medications, Sjogren's syndrome, vitamin A deficiency, and the like); mechanical trauma (e.g., fingernail scratch, edge of contact lens, foreign body in the lid/fornices; trichiasis, a common eyelid problem, wherein eyelashes grow inwards toward the eye, rub against the cornea, the conjunctiva, and the inner surface of the eyelids, and irritates the eye; distichiasis, an eyelash abnormality where an extra set of eyelashes have grown in the wrong place on the eyelids; neurotrophic diseases causing incomplete lid closure (such as palsy of the 7th cranial nerve or Bell's palsy); restrictive eyelid diseases; proptosis/exophthalmos, characterized by the bulging or protruding of one or both eyes from their natural position; decreased consciousness in drug abuse or comatose state; blepharoplasty (a surgical rejuvenating procedure that may be performed on the upper and/or lower eyelids) that overcorrects ptosis; lagophthalmos characterized by incomplete or abnormal closure of the eyelids; exposure during long surgeries under general anesthesia; ultraviolet burns (e.g. welding, prolonged exposure to the sun or reflective surfaces); limbal stem cell deficiency (failure to regenerate epithelial cells may be due to chronic contact lens wear, chemical burns, a history of ocular surgery, ocular autoimmune degenerations, and the like); topical anesthetic abuse; neurotrophic keratopathy (e.g., corneal hypoesthesia or anesthesia caused by damage to the trigeminal nerve, HSV, VZV, diabetes, topical drop toxicity, and the like); and infection (bacteria and other organisms can invade the cornea through the epithelium causing epithelial defects or ulcers).

Embodiments disclosed herein pertain to utilization of mitochondrial transplantation for treatment of damaged endothelium cells of the cornea.

Human corneal endothelial cells possess very limited proliferative capacity, so when cells are lost, tissue repair relies mainly on the remaining cells spreading and migrating to cover the defect. This migratory process is strongly dependent on mitochondrial activity, as directed movement requires localized ATP generation to drive actin remodeling, focal-adhesion turnover, and cytoskeletal contractility. In many corneal pathologies, chronic oxidative stress disrupts these mitochondrial functions, thereby impairing the respiratory chain, reducing membrane potential, and elevating mitochondrial ROS. As mitochondrial efficiency declines, the energetic support needed for coordinated migration diminishes, resulting in inadequate closure of intercellular gaps.

The present inventors have demonstrated that transplantation of exogeneous mitochondria to “wounded” (scratched) cultures of corneal endothelial cells (hCEC-12 cell line) and to cultures of primary human corneal endothelial cells isolated from harvested human corneas, enhanced cell migration, independent of proliferation, indicating a pro-migratory effect (see Example 11 herein).

In some embodiments, a disclosed method is applied in vivo, namely, in treating a subject afflicted with damaged corneal endothelium.

In some embodiments, transplantation of exogeneous mitochondria is conducted ex vivo in corneal endothelial cells (hCEC) harvested from damaged corneal tissue, optionally for utilization in corneal regenerative therapy.

It is shown in Example 12 herein that corneal endothelia harvested from human corneas (rejected for corneal transplantation) could be expanded ex vivo following mitochondrial transplantation. Replenishing damaged hCEC cells with healthy viable mitochondria elevated ATP productions (Example 13), remedied oxidative stress-associated cellular reorganization (Example 14), and promoted activation of the ERK and JNK pathways that play a critical role in mediating enhanced wound-healing response (Examples 15 and 16). Thus, principally, defects in corneal endothelium can be corrected or modified ex vivo by harvesting CEC cells from a damaged corneal endothelium and injecting the cured cells back into the anterior chamber of the subject. The injected CEC cells may be allowed to adhere to Descemet's membrane using a positioning maneuver (e.g., face-down positioning). In this manner, mitochondrial transplantation in the culturing step serves as a successful means for restoration of corneal clarity and healing improvement in patients with endothelial dysfunction.

Damage to the corneal endothelium may be facilitated by any one or more of the causes mentioned herein, for example, oxidative stress, age-related decline in endothelial cell density, inherited or non-inherited degenerative disease, surgical trauma, injury or ocular trauma, inflammation, infection, increased intraocular pressure (IOP), contact lens-related hypoxia, contact lens overuse, a corrosive agent, exposure to a chemical or toxin, chemical or thermal burns, reduced oxygen or nutrient supply to the cornea, a systemic disease and cryotherapy or laser procedures.

In some embodiments, the damage to the corneal endothelium is caused by oxidative stress.

In some embodiments, the damage to the corneal endothelium is caused by Fuchs endothelial corneal dystrophy.

Based on the observation by the present inventors that exogenous mitochondria enhance activation of the ERK and JNK pathways in hCEC-12 cells, it is envisaged that the mitochondrial effect in corneal wound healing is augmented or improved by co-application of modulators of MAPK pathways, for example, active agents that promote activation of ERK and JNK pathways.

The Rho-associated protein kinases (ROCK) pathway regulates cytoskeletal organization (actin-myosin contraction), cell shape and adhesion, cell migration, proliferation and survival, wound healing and regeneration. The ROCK pathway does not operate in isolation. It interacts (“crosstalks”) with MAPK pathways, including ERK, JNK and p38 MAPK, which is why ROCK is described as a modulator of MAPK signaling. Hence, changing ROCK activity indirectly alters MAPK signaling.

ROCK modulators are agents that activate or inhibit Rho-associated protein kinases ROCK1 and ROCK2. ROCK modulators affect how cells move, survive, and regenerate. Modulating ROCK can indirectly regulate MAPK-dependent outcomes, such as cell survival, regeneration, inflammation and apoptosis. This is particularly important in endothelial cells, neuronal cells, corneal endothelial cells and stem/progenitor cells. For example, ROCK inhibition can enhance ERK signaling in some contexts (e.g., promoting cell proliferation or migration) and/or suppress stress-activated pathways (e.g., JNK or p38) in others. Thus, in some embodiments, co-application of exogenous mitochondria and one or more ROCK modulators, and optionally a modulator of MAPK pathway, will improve or augment the beneficial mitochondrial effect in corneal wound.

Common ROCK modulators include ROCK inhibitors (e.g., Y-27632, fasudil, ripasudil) and less commonly, ROCK activators (often indirect, via RhoA signaling).

Further corneal (epithelium and/or endothelium) pathologies treatable by a disclosed method include corneal abrasion, recurrent erosion syndrome, and corneal dystrophies involving mitochondrial dysfunction in their pathogenesis, such as certain types of epithelial basement membrane dystrophies associated with erosion.

In corneal dystrophies, foreign material builds up in one or more of the five layers of the cornea. The material may cause the cornea to lose its transparency, leading to loss of vision or blurred vision. Most corneal dystrophies affect both eyes. They progress slowly and run in families.

Recurrent corneal erosion (RCE) syndrome is a common, recurrent dystrophy caused when the epithelium layer loosens from the layer underneath (Bowman's membrane). This abnormal epithelial adhesion to the underlying basal lamina may cause the sudden onset of mild to severe pain in the eye, light sensitivity, blurred vision, tearing and the feeling of a foreign object in the in the eye, typically upon awakening.

A corneal abrasion (also called scratched eye or scratched cornea) is an eye injury that causes significant discomfort, photophobia, and erythema. This may occur spontaneously or when there is a disruption of the corneal epithelium caused by contact lenses.

The state-of-the-art treatment or management of the above-mentioned corneal pathologies may be either medical (e.g., lubricating eye drops, antibiotic ointments) or surgery to remove the damaged epithelium and promote healing. The transplantation of healthy mitochondria can restore cellular respiration and energy production, leading to faster cell proliferation and improved wound healing. Furthermore, as demonstrated herein, this approach has minimal adverse effects, making it a potentially safe and effective treatment for corneal injury.

In some embodiments, the administered mitochondria in accordance with a contemplated method, are freshly isolated mitochondria.

An effective amount or a therapeutically effective amount of exogenous, e.g., fresh, mitochondria, as referred to herein, is a quantity, e.g., number or concentration of isolated mitochondria, which is sufficient to achieve a desired effect in a subject being treated. An effective amount of isolated mitochondria can be administered in a single dose, or in several doses, for example, once or several times, during a course of treatment. For example, mitochondrial transplantation may be provided to a patient only one time, for example, in treatment of an acute ophthalmic pathology. Alternatively, or additionally, mitochondrial transplantation may be provided, for example, once a week, once a month, once in two months, once in 6 months, once a year, once in two years, and the like, for example, when treating a chronic pathology of the eye. However, the effective amount of the isolated mitochondria will depend on the state of the mitochondria applied, the subject being treated, the severity and type of the affliction, and the manner of administration of the transplant.

In some embodiments, the therapeutically effective amount of purified, isolated mitochondria administered, e.g., directly to the eye, in accordance with a contemplated method, is in the range of from about 10⁴ to about 10¹⁰ dose per administration, for example, from about 10⁴ to about 10⁵, from about 10⁵ to about 10⁶, from about 10⁵ to about 10⁷, from about 10⁶ to about 10¹⁰, from about 10⁶ to about 10⁸, from about 10⁶ to about 10⁷, from about 10⁷ to about 10¹⁰, from about 10⁷ to about 10⁹, from about 10⁸ to about 10¹⁰, from about 10⁸ to about 10⁹, from about 10⁹ to about 10¹⁰ or more, of isolated, e.g., fresh or preserved mitochondria.

In exemplary embodiments, fresh, isolated mitochondria are administered intravitreally in an effective amount of from about 10⁶ to about 10⁷mitochondria, e.g., in a single dose.

In systemic administration, a therapeutically effective amount of exogenous mitochondria is in the range of from about 0.1 to about 1.5 mg/kg body weight, for example, from about 0.1 to about 0.5, from about 0.2 to about 0.5, from about 0.3 to about 0.6, from about 0.3 to about 0.8, from about 0.4 to about 0.5, from about 0.4 to about 0.7, from about 0.5 to about 0.6, from about 0.5 to about 0.8, from about 0.6 to about 0.9, from about 0.6 to about 1.1, from about 0.8 to about 1.2, from about 0.8 to about 1.0, or from about 0.9 to about 1.5 mg/kg body weight.

In some exemplary embodiments, the amount of exogenous isolated, e.g., fresh, mitochondria systemically administered is from about 0.4 to about 0.7 mg/kg body weight, or about 0.5 mg/kg body weight.

As appreciated by a skilled person, excess mitochondria transplantation is to be avoided because it may cause inflammation and fibrosis of the retina and vitreous.

Treating a disease, as referred to herein, means ameliorating, inhibiting the progression of, delaying worsening of, and even completely preventing the development of a disease. Treatment refers to a therapeutic intervention that ameliorates a sign or symptom of a disease or a pathological condition after it has begun to develop. In particular examples, however, treatment is similar to prevention, except that instead of complete inhibition, the development, progression or relapse of the disease is inhibited or slowed. Preventing a disease refers to inhibiting the full development of a disease, for example inhibiting vision loss due to ischemic insult.

“Administration”, as referred to herein, is introduction of the isolated mitochondria or a formulation comprising it, as defined herein, into a subject by a chosen route. Administration of the isolated mitochondria or of a formulation can be by any route known to one of skill in the art, and as appropriate for the particular condition and location under treatment. Administration can be local or systemic.

Examples of local administration pertaining to the present disclosure include, but are not limited to, intravitreal injection, intra ocular-muscle administration, injection into the anterior chamber, injection into the suprachoroidal space, injection into the limbus, retro bulbar injection, sub-tenon injection, subconjunctival injection, intracorneal injection, and topical application to the surface of the eye (for example, by designated eye drops). In addition, local administration includes routes of administration typically used for systemic administration, for example by directing intravascular administration to the arterial supply of the eye. Thus, in particular embodiments, local administration includes intra-arterial administration and intravenous administration when such administration is targeted to the vasculature supplying the eye.

All these routes of administration expose different cells to the exogenous mitochondria: in intravitreal administration (e.g., injection), RGC and maybe other cells in the inner retina are exposed to the exogenous mitochondria; in anterior chamber injection, corneal endothelium and other cells in the anterior chamber are exposed to the exogenous mitochondria; in suprachoroidal or subretinal space injection, choroid cells, RPE and photoreceptors are exposed to the exogenous mitochondria; in application to the surface of the eye, corneal epithelium is exposed to the exogenous mitochondria; and in intravenous administration, exogenous mitochondria can reach any ocular site if the retinal blood barrier does not exist or if it is defected such as in diabetic retinopathy.

In some embodiments, exogenous mitochondria are administered via intravitreal injection (see FIG. 1B). Alternative or additional modes of ocular administration of mitochondria are schematically presented in FIG. 1C.

Systemic administration includes any route of administration designed to distribute an active compound, e.g., isolated mitochondria, widely throughout the body via the circulatory system. Thus, systemic administration includes, but is not limited to intra-arterial and intravenous administration. Systemic administration also includes subcutaneous administration, or intramuscular administration, when such administration is directed at absorption and distribution throughout the body by the circulatory system.

### Pharmaceutical Compositions and Formulations

The present disclosure, in an aspect thereof, is related to a pharmaceutical composition comprising isolated mitochondria and a pharmaceutically acceptable excipient. In some embodiments, the composition is a formulation for pharmaceutical administration and comprises a pharmaceutically acceptable carrier.

The term “pharmaceutical composition”, as used herein, refers to a composition essentially comprising isolated mitochondria, which may be adjusted for medicinal utilization such as therapeutic or diagnostic utilization. “Formulation”, as used herein, refers to any mixture of different components or ingredients, at least one of which is mitochondria, e.g., autologous or allogeneic freshly isolated and purified mitochondria, prepared in a certain way, i.e., according to a particular formula so as to be applicable for administration to a subject. Such a formulation is termed herein “mitochondrial formulation”. For example, mitochondrial formulation may be formulated for mitochondrial transplantation, and may include mitochondria combined or formulated together with, for example, one or more carriers, excipients, stabilizers and the like. Usually, a mitochondrial formulation comprises one or more pharmaceutically and physiologically acceptable carriers, which can be administered to a subject (e.g., human or non-human subject) in a specific form, such as, but not limited to, infusion, injection or eye drops. The mitochondrial formulation may further comprise other active agents such as anti-inflammatory agents, antibiotics and the like. For example, a mitochondrial formulation may comprise an agent which promotes mitochondrial biogenesis, such as erythropoietin (EPO) or a salt thereof, for example, recombinant human erythropoietin or isolated human erythropoietin.

As used herein, the terms “pharmaceutically acceptable”, and “physiologically acceptable” are interchangeable and mean approved by a regulatory agency of the Federal or a state government or listed in the U.S. Pharmacopeia or other generally recognized pharmacopeia for use in animals, and more particularly in humans. These terms include formulations, molecular entities, excipients, carriers and compositions that do not produce an adverse, allergic or other untoward reaction when administered to an animal, or a human, as appropriate. For human administration, preparations should meet sterility, pyrogenicity, general safety and purity standards as required by, e.g., the U.S. Food and Drug Administration (FDA) agency, and the European Medicines Agency (EMA).

Herein the term “excipient” refers to an inert substance added to a pharmaceutical composition or formulation to further facilitate process and administration of the active ingredients. “Pharmaceutically acceptable excipients”, as used herein, encompass approved preservatives, antioxidants (e.g., ascorbic acid (vitamin C) or a salt thereof; cysteine or a cysteine derivative; lipoic acid; uric acid; carotenes; α-tocopherol (vitamin E); and ubiquinol (coenzyme Q)), surfactants (e.g., Tween®-20, Tween®-40, Tween®-60 and Tween®-80), a buffer (e.g., citrate buffer, acetate buffer, sodium acetate buffer, tartrate buffer, Tris buffer, glycine buffer, sodium buffer, sodium hydroxide buffer, or a mixture thereof), coatings, isotonic agents, absorption delaying agents, carriers and the like, that are compatible with pharmaceutical administration, do not cause significant irritation to an organism and do not abrogate the biological activity and properties of a possible active agent. Physiologically suitable carriers in liquid formulations may be, for example, solvents or dispersion media. The use of such media and agents in combination with mitochondria is well known in the art.

A disclosed formulation may be used for mitochondrial transplantation in accordance with any of the methods described herein.

### Kits

In still a further aspect, the present disclosure relates to a kit comprising isolated mitochondria or a formulation comprising isolated mitochondria, as defined herein, and, optionally, instructions and means for administration of the mitochondria and/or the formulation to a subject in need thereof.

A contemplated kit is useful for treatment of mitochondrial dysfunction, mitochondrial destruction or depletion in the eye of a subject and any ophthalmic disease, disorder or condition and/or any ocular manifestation of a disease or condition associated with mitochondrial dysfunction in the eye as described herein.

In some embodiments, a contemplated kit is useful for treatment of ischemic insult in the eye.

As used herein the term “about” refers to ±10 %.

The terms “comprises”, “comprising”, “includes”, “including”, “having” and their conjugates mean “including but not limited to”.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this disclosure may be presented in a range format. It should be understood that the description in range format is merely for convenience and brevity and should not be construed as an inflexible limitation on the scope of the description. Accordingly, the description of a range should be considered to have specifically disclosed all the possible subranges as well as individual numerical values within that range. This applies regardless of the breadth of the range.

Various embodiments and aspects of the present disclosure as delineated hereinabove and as claimed in the claims section below find experimental support in the following examples.

### EXAMPLES

Reference is now made to the following examples, which together with the above descriptions illustrate some embodiments of the disclosure in a non-limiting fashion.

Generally, the nomenclature used herein, and the laboratory procedures utilized in the present disclosure include molecular, chemical, biochemical, microbiological and/or recombinant DNA techniques. Such techniques are thoroughly explained in the literature. See for example, Guide to Research Techniques in Neuroscience (Second Edition), Matt 2015; Elsevier's Integrated Review Biochemistry (Second Edition), 2012. Other general references are provided throughout this document. The procedures therein are believed to be well known in the art and are provided for the convenience of the reader. All the information contained therein is incorporated herein by reference.

### Materials and Methods

### Animals

Male C57BL/6 mice aged 8-10 weeks and weighing 20-25 gr were used. The mice were housed in the Kaplan Medical Center animal facility at 21-22° C. with 12/12-hour light-dark cycles.

All experiments and animal care procedures were conducted in accordance with the Association for Research in Vision and Ophthalmology (ARVO) statement for use of animals in ophthalmic and vision research, under the supervision of a health authority-accredited staff member for animal care and management in Kaplan Medical Center. In addition, the research protocol was approved by the national animal care and use committee (approval ID IL-18-8-241).

### Cell Culture

Human neuroblastoma SH-SY5Y cells, originally derived from a metastatic bone tumor biopsy and differentiated to a neuron-like phenotype, are herein referred to as “neuronal cells”. Human adult retinal pigment epithelial cell line-19 (ARPE-19), are a spontaneously arising retinal pigment epithelia cell line. Both SH-SY5Y and ARPE-19 cells were grown in DMEM-F12 media, supplemented with 10% FBS, 1% glutamine, 200 U/ml penicillin and 200 mg/ml streptomycin. Human corneal endothelial cells (hCECs) were isolated from the limbus of human corneas, meaning that they originate from limbal cells. hCECs were grown in DMEM media, supplemented with 5% FBS, 1% glutamine, 200 U/ml penicillin and 200 mg/ml streptomycin. Cells were maintained at 37° C. in a humidified atmosphere containing 95% air and 5% CO₂.

### Mitochondrial Isolation

Mitochondria were either isolated from ARPE-19 cell-line for in-vitro studies, or from mouse liver for in-vivo experiments, according to published protocols (see, for example, Wieckowski et al., 2009, Nature Protocols 4:1582-1590; and Frezza et al., 2007, Nature Protocols 2:287-295). For liver mitochondria isolation, before each experiment, and following overnight fasting, C57BL/6 mice were anesthetized using CO₂ and euthanized by cervical dislocation. The liver was harvested and immediately washed with ice-cold IB-1 buffer (225 mM mannitol, 75 mM sucrose, 30 mM Tris-HCl, 0.5% fatty acid free bovine serum albumin (FA-free BSA), 0.5 mM EGTA, pH 7.4) and weighed. Then, the liver was rinsed in IB-3 buffer (225 mM mannitol, 75 mM sucrose, 30 mM Tris-HCl, pH 7.4), cut into small pieces using scissors, transferred to a glass potter in IB-1 buffer and homogenized gently 10 times to break cellular membranes. An aliquot of 100 μl was kept aside and served as an ‘input’ control for Western blot (WB) analysis. The homogenate was centrifuged once or twice at 750 g for 10 min at 4° C., and the supernatant was separated and centrifuged at 10,000 g for additional 10 min at 4° C. The supernatant containing the cytosolic fraction was kept for WB analysis. The pellet, which contained the mitochondria was gently re-suspended in IB-2 buffer (same as IB-1 but without EGTA) and centrifuged again at 10,000 g for 10 min at 4° C. The supernatant was discarded, and the mitochondrial pellet was kept on ice and used within 3-4 hours.

For in-vitro studies, approximately 250×10⁶ ARPE-19 cells were collected from 8×15 cm dishes and mitochondria were isolated in a similar way.

Typically, 4-10 mg of mitochondria, ~1×10⁹, were obtained from ~1 gr mouse liver tissue, and 0.2-1 mg, ~1×10⁹ mitochondria were obtained from 8 confluent 150 mm tissue culture plates of human ARPE-19 cells (~12×10⁷ cells/plate).

### Western Blot

Western blotting, as referred to herein, also known as immunoblotting (because antibodies are used for specifically detecting their antigens) or protein blotting, is a core technique useful in separating and identifying proteins from a complex mixture of proteins extracted from cells. The term “blotting” refers to the transfer of a biological sample from a gel to a membrane and its subsequent detection on the surface of the membrane. The technique is based on three main steps: (1) separation of cell extract (or homogenate) proteins by molecular weight (and thus by type) through gel electrophoresis, for example, using agarose gel having a lower acrylamide concentration such as sodium dodecyl sulfate polyacrylamide gel electrophoresis (SDS-PAGE); (2) transfer to a solid support (membrane) such as nitrocellulose or polyvinylidene difluoride (PVDF), which produces a band for each protein; and (3) identifying specific desired proteins by incubating the membrane with antibodies specific to the proteins of interest (herein referred to as “primary antibodies”), and labelling the antibodies by the use of secondary, e.g., fluorescently or otherwise labelled, antibodies. The bound antibodies may be detected, for example, by developing the film and/or by using a designated analysis kit. As the antibodies only bind to the protein of interest, only one band should be visible for a specific protein. The thickness of the band corresponds to the amount of protein present. Multiplexing fluorescent western blotting enables multiple proteins to be detected and quantified in a single sample. Direct protein abundance comparisons and normalization may be conducted against a control, for instance a protein encoded by a housekeeping gene.

In the present disclosure, 30 μg of mitochondrial proteins were resolved (separated) on SDS-PAGE gel and transferred to nitrocellulose membrane and, after reaction with the desired antibodies, were developed using EZ-ECL chemiluminescence detection kit (Biological Industries). This kit is applied to membrane-immobilized proteins conjugated with horseradish peroxidase (HRP) directly or indirectly. In the presence of hydrogen peroxide (H₂O₂), HRP catalyzes the oxidation of cyclic diacylhydrazides such as luminol and excites them. The intermediate reaction product, which is in its excited state, decays to the ground state by emitting light.

The following primary antibodies were used: mouse anti cox-4 (bs-10257R, Bioss Antibodies, 1:200) specific to a mitochondrial protein, and rabbit anti β-actin (A5441, Sigma-Aldrich, 1:5000) specific to a cytosolic, non-mitochondrial protein. Secondary antibodies used were HRP-conjugated secondary antibodies (Jackson ImmunoResearch).

### ATP Production

Isolated mitochondria were diluted 1:100 in respiration buffer (~0.5 mg/ml mitochondria per vial). Succinate (5 mM) was added as an electron donor to all vials. Basal ATP production, ADP-induced respiration and mitochondrial ATP production (150 μM ADP as substrate) were determined using ATPlite™ luminescence assay kit (PerkinElmer®, US), according to manufacturer instructions. The ATPlite™ assay system is based on production of light caused by reaction of ATP with added D-luciferin in the presence of luciferase. The emitted light is proportional to the ATP concentration (within certain limits).

Oligomycin (10 μM), an inhibitor of ATP synthase, was used as a control to validate mitochondrial-dependent ATP production.

Luminescence was measured by a microplateplate reader (Tecan, Switzerland).

### Mitochondrial Respiration

Oxygen consumption studies were performed using a Clark type electrode (Hansatech Instruments, UK) as described in Rustin et al. (Rustin et al., 1994, Clinica chimica acta; International J Clinical Chemistry 228:35-51), and according to the manufacturer instructions. Briefly, oxygen dissolved in the reaction vessel of a liquid-phase system was detected polarographically by the S1 Clark-Type electrode. This oxygen electrode disc comprises a relatively large (2 mm diameter) platinum cathode and a concentric silver anode immersed in and linked by an electrolyte solution, also termed herein “respiration buffer”. The electrodes are protected by a thin PTFE (Teflon) membrane which is permeable to oxygen. When a small voltage is applied across these electrodes such that the platinum is made negative with respect to the silver, the current which flows is at first negligible and the platinum becomes polarized (i.e., it adopts the externally applied potential). As this potential is increased to 700 mV, oxygen is reduced at the platinum surface, initially to hydrogen peroxide H₂O₂. The polar platinum electrode discharges as electrons are donated to oxygen, which acts as an electron acceptor. The current which then flows is stoichiometrically related to the oxygen consumed at the cathode. It is converted to a digital signal and recorded by an electrode control unit.

Isolated mitochondria (0.4-0.6 mg) were measured in a respiration buffer comprising 225 mM mannitol, 75 mM sucrose, 2 mM HEPES, 5 mM KH₂PO₄, 5 mM MgCl₂, 1 mM EDTA and 0.1% FA-free BSA, pH 7.4, at 37° C. Succinate (5 mM) was used as a substrate, which feeds its electrons into the respiratory chain via complex II (state II respiration). Rotenone (2 μM) was added to inhibit complex I activity and to prevent succinate-driven reverse electron transfer. To induce state III (complex III) respiration, 150 μM ADP were added to the chamber, and the oxygen consumption was recorded until ADP levels decreased (state IV) and the respiration returned to a rate comparable to state II.

### Optic Nerve Crush (ONC) Retinal Ischemic Model

Mice were placed under general anesthesia by intraperitoneal injection of combined ketamine/xylazine (80 mg/kg and 8 mg/kg, respectively), supplemented by topical ophthalmic anesthesia (proparacaine hydrochloride 0.5%). For inducing retinal ischemia, the right optic nerve was crushed by applying forceps at 2.5 to 3.0 mm posterior to the globe for 7 seconds; this procedure was repeated three times (see, further description, for example, in Rappoport et a., 2013, Investigative Ophthalmology & Visual Science 54:8160-8171). Three groups of mice were tested: (1) mice experiencing optic nerve crush followed immediately by a single intravitreal injection of 1 or 10 million fresh mitochondria isolated from mouse liver, suspended in 10 μl PBS; (2) mice experiencing ONC followed immediately by a single intravitreal injection of 10 μl PBS. These mice served as a sham (false) control; and (3) mice that were injected with 1 or 10 million mitochondria without performing ONC. The left eye of each mouse in all groups served as a control. The intravitreal injections of mitochondria were performed in a standardized manner under direct ophthalmoscopic control with an operating microscope (Zeiss S3, Germany). Mice were euthanized by carbon dioxide 1 or 14 days after injury. For fixation, 4% paraformaldehyde was perfused via the vascular circularity system, the eyes were enucleated and kept in formalin until used.

### Whole Mount Retina Preparation

The retina is a thin sheet of nervous tissue that lines the back of the eye. It processes the optical image of the visual field, transforming light energy into electrical signals (i.e., phototransduction) to form a neural image interpretable by the rest of the brain. Retinal ganglion cells (RGCs) populate the ganglion cell layer (GCL) of the retina (see FIGS. 2A-2B depicting a schematic presentation (2B), and optical coherence tomography (OCT) image (2A) of layers of the retina). Retinal ganglion cells are the bridging neurons that connect the retinal input to the visual processing centers within the central nervous system. There is a remarkable diversity of RGCs and the various subtypes have unique morphological features, distinct functions, and characteristic pathways linking the inner retina to the relevant brain areas.

Retina is one of the most easily accessible parts of the central nervous system in vertebrates. The retinal wholemount technique is a useful method of assessing the structure, arrangement and sampling of retinal neurons in vertebrate eyes. Numerous visual parameters can be determined with the wholemount technique, including spatial resolving of ganglion cells spacing and integrity. Using wholemounts, total absolute counts of RGC number, and acceptable estimates of percentage differences in RGC numbers between treatment and control groups may be calculated.

Wholemount retina were prepared from 4% paraformaldehyde (PFA) fixed eyes according to the protocol described by Ivanova et al. (Ivanova et al., 2013, J. Visualized Experiments, JoVE:e51018). In brief, a mouse was euthanized, and his eyes were enucleated, washed 3 times with 1 ml PBS and placed in a 6 cm dish in PBS. Under a microscope (Zeiss S3, Germany), the cornea, the lens and the vitreous were removed. Then, a small cut was made underneath the RPE layer, and the neuro-retina was gently separated from the eyecup. When the entire retina was used, small incisions were made from the periphery, halfway to the optic nerve. These incisions helped in making a flat preparation. Retinae were flattened onto glass slides (SuperFrost™ slides of Thermo Scientific™).

### Mitochondrial Staining and Uptake Evaluation

For in-vitro mitochondrial uptake assessment, 30,000 SH-SY5Y cells were seeded on cover slips in a 24 wells plate. On the next day, isolated mitochondria from ARPE-19 cells were labeled (stained) with 200 nM Mito Tracker® Red CMXRos dye (M7512, Invitrogen™ Molecular Probes™ CA, USA), herein designated “MTR”. MTR is a red-fluorescent dye that accumulates and stains mitochondria in live cells, depending upon the mitochondrial transmembrane potential (Δψₘ), thereby being indicative of mitochondrial function. The dye is well-retained after aldehyde fixation. The MTR labeled mitochondria (about 2.5×10⁶) were added to the SH-SY5Y cells. Twenty-four hours later, cells were fixed with 4% PFA for 10 mins, washed with PBS, and subjected to immunostaining as described herein below.

For in-vivo uptake evaluation, mouse liver isolated mitochondria were labeled (stained) with 200 nM MTR, and 1 or 10 million of the labeled mitochondria were injected intravitreally immediately following optic nerve crush (ONC). Twenty-four hours after injection, mice were euthanized, perfused with 4% PFA, and their eyes were enucleated. Whole mount retina or paraffin embedded eye sections were prepared and subjected to immunostaining as described herein below.

### Histological and Immunostaining

Histological staining of cells, most of which are colorless and transparent, usually employs a dye that stains some of the cell components a bright color, together with a counterstain that stains the rest of the cell a different color. Acidic dyes react with cationic or basic components in cells (also termed “acidophilic”) such as proteins and other components in the cytoplasm. Basic dyes react with anionic or acidic components in cells (also termed “basophilic”), for example, nucleic acids. The staining system termed “H&E staining” contains two dyes: the basic dye hematoxylin and the acidic dye eosin. Eosin stains acidophilic structures (e.g., cytoplasmic proteins, cytoplasmic filaments in muscle cells, intracellular membranes, and extracellular fibers) red or pink. Haematoxylin is used in combination with aluminum ions (Al³⁺) for staining basophilic structures (e.g., the nucleus and parts of the cytoplasm that contain RNA (e.g., ribosomes and the rough endoplasmic reticulum)) purplish blue.

For histological staining, 4 μm paraffin embedded eye sections were stained with H&E according to standard staining protocol for light microcopy assessment (see, for example, Rappoport et a., 2013, Investigative Ophthalmology & Visual Science 54:8160-8171). RGCs were counted using ImageJ software (in a ×40 magnification field) for three consecutive sections of every 7 to 10 slides of each eye, and the mean number was calculated.

Immunostaining or immunohistochemistry (IHC) images discrete components in tissues by using appropriately labeled antibodies to bind specifically to their target antigens in situ. IHC makes it possible to visualize and document the high-resolution distribution and localization of specific cellular components within cells and within their proper histological context. IHC methodology includes two main steps: sample preparation and sample staining. Tissue preparation or fixation is essential for the preservation of cell morphology and tissue architecture. Traditional IHC is based on the immunostaining of thin sections of tissues attached to individual glass slides. Immunostained slides are prepared and processed either manually or automatically for high-throughput sample preparation and staining. Samples can be viewed by either light or fluorescence microscopy.

Immunostaining of wholemount retinae was performed as described, for example, in Mead et al., 2014, PloS one 9:e110612. Briefly, retinae were permeabilized in 0.5% Triton x-100 in PBS for 15 min at −70° C. and then washed at room temperature with 0.5% Triton x-100 for a further 15 min. Retinae were incubated with primary antibodies diluted in wholemount antibody diluting buffer (wADB; 2% bovine serum albumin, 2% Triton x-100 in PBS) overnight at 4° C., and the following day were washed 3×10 min in PBS and incubated with secondary antibodies in wADB for 2 h at room temperature. After 2 h, retinae were washed for 3×10 min in PBS and mounted, the ganglion cell layer (GCL) uppermost, on glass slides in mounting medium (GBL labs), and cover slips were applied. Retina was immunostained for Tuj1, a neuron-specific class III β-tubulin, for marking ganglion cells. Other antibodies employed are detailed below.

For immunostaining of paraffin sections, de-paraffinization and antigen retrieval were performed according to standard protocol, before applying immunostaining.

The following antibodies were used: mouse anti-cox4 (bs-10257R, Bioss Antibodies, MA, USA 1:200), rabbit anti-β-actin (A5441, Sigma-Aldrich, 1:50), chicken anti-β-tubulin III/Tuj1(GTX85469, GeneTex, CA, USA), and fluorescent secondary antibodies (Jackson ImmunoResearch, PA, USA).

Fluorescent images were taken using fluorescent microscope (Nikon TS 100, Japan). Confocal images were observed under confocal laser-scanning microscope Zeiss LSM 510.

The fluorescent stain 4′,6-diamidino-2-phenylindole (DAPI) which binds preferentially to the AT-rich regions of dsDNA was applied for visualizing nuclear DNA in both living and fixed cells (DAPI staining). This staining quantifies the number of nuclei and assesses gross cell morphology. In embodiments described herein, DAPI staining was used to label nuclei.

### Flow Cytometry

Flow cytometry is an analytical cell-biology technique that utilizes light to count and profile cells in a heterogenous fluid mixture. Flow cytometry analysis was used to evaluate mitochondrial internalization into neurons. For assessing mitochondrial internalization, 5×10⁴ SH-SY5Y cells were seeded in 12 wells plates in 1 ml media and incubated over night at 37° C. and 5% CO₂. On the next day, mitochondria isolated from ARPE-19 cells, were stained with 200 nM Mito Tracker® Green-Fixable Mitochondrion dye (M-7514, Invitrogen™ Molecular Probes™ CA, USA), termed herein “MTG”, and added to the SH-SY5Y neuronal cells for 18 h. SH-SY5Y cells of one well were stained for endogenous mitochondria with 500 nM MTG and served as a positive control. These cells were not transplanted with exogenous mitochondria. For staining, the cells were incubated with the dye, which passively diffused across the plasma membrane and accumulated in mitochondria. Mitochondria in cells stained with nanomolar concentrations of MTG exhibit bright green, fluorescein-like fluorescence. The MTG dye is nonfluorescent in aqueous solutions and becomes fluorescent only once it accumulates in the lipid environment of mitochondria. Hence, background fluorescence is negligible, enabling to clearly visualize mitochondria in live cells immediately following addition of the stain, and without a wash step.

Cells were trypsinized and collected into fluorescence-activated cell sorting (FACS) tubes, centrifuged at 1,400 g for 5 min and resuspended in PBS enriched with 1% fetal bovine serum (FBS). The mean intensity of the cellular MTG fluorescence was measured on a CytoFlex instrument (Beckman Coulter, USA) at excitation/emission wavelengths of 488/530 nm. Cell viability was simultaneously evaluated by staining with DRAQ7™ (BD Biosciences, US) and measuring its fluorescence at excitation/emission wavelengths of 488/644 nm. Dead cells (DRAQ7 positive cells) were excluded from the analysis. DRAQ7™ (Deep Red Anthraquinone 7) is a far-red fluorescent DNA dye, which is cell impermeable and is used to stain dead cells only in viability tests. DRAQ7™ has an excitation wavelength maximum of 599/644 nm but can also be suboptimally excited by the 488 nm wavelength laser.

Data were analyzed using CytoExpert 2.2 software and presented as the mean fluorescence intensity of the live cells.

### Optical Coherence Tomography (OCT)

Optical coherence tomography (OCT) is a non-invasive imaging technique that uses low-coherence light to generate detailed, cross-sectional images of the eye, particularly the retina. It works by directing a beam of near-infrared light into the eye and measuring the tiny delays in the light reflected from different retinal layers. These reflections are analyzed using interferometry, allowing the system to reconstruct the depth and structure of the tissue with micrometer-level resolution.

Because the light scans across the retina point by point, OCT builds a layered image that shows the thickness, contour, and integrity of each retinal layer. Modern OCT systems acquire these scans quickly and can also compile them into three-dimensional maps of the retina and optic nerve. This makes OCT invaluable for diagnosing and monitoring conditions such as macular degeneration, glaucoma, diabetic macular edema, and other disorders that alter retinal structure. It provides precise, repeatable measurements without touching the eye.

### Fundus Imaging

Fundus imaging is a non-invasive technique used to photograph and document the interior surface of the eye, known as the fundus, which includes the retina, optic nerve head (where the optic nerve exits the eye), macula (the central area responsible for sharp, detailed vision), and retinal blood vessels.

A fundus camera (specialized for the fundus) uses a combination of low-intensity flash illumination and magnifying optics to capture a wide, clear view of these internal structures. The resulting images allow to evaluate retinal health, detect abnormalities, and monitor disease progression over time. Fundus imaging is widely used to diagnose and follow conditions such as diabetic retinopathy, age-related macular degeneration, glaucoma, retinal detachments, and vascular disorders.

### Example 1

### Isolation and Purification of Active Mitochondria

Mitochondria for transplantation were isolated form mouse liver and their purity and quality were evaluated by Western blotting as described in Materials and Methods.

As shown in FIG. 3A, the purified mitochondrial fraction was enriched with the mitochondrial protein cytochrome c oxidase (Cox4) and was depleted of the cytosolic marker protein B-actin, which appeared in the cytosolic fraction (cyto), demonstrating a pure mitochondrial preparation. Both markers appeared in the input sample before fractionation.

Next, mitochondrial function, i.e., respiration, was assessed by measuring oxygen consumption in the purified mitochondria using a Clark oxygen electrode, following addition of a substrate (succinate) and ADP, or rotenone (an inhibitor of succinate-driven respiration), as described in Materials and Methods. The results presented in FIG. 3B demonstrate that the mitochondria were indeed functional.

Mitochondrial activity was further confirmed by measuring ATP levels, and the results are presented in FIG. 3C. Mitochondria alone (Mito) showed base line ATP production. Addition of ADP substrate (Mito-ADP) substantially increased ATP levels, while introduction of oligomycin (Mito-Olm), an ATP-synthase inhibitor, dramatically reduced ATP levels, indicating appropriate ATP production capability of the isolated mitochondria. Finally, isolated mitochondria were labeled with MitoTracker® Green (MTG) Fixable Mitochondrion (FM) dye (M-7514, Invitrogen™ Molecular Probes™ CA, USA), which stains mitochondria regardless of their membrane potential, as well as with MitoTracker® Red CMXRos (MTR dye) (M7512, Invitrogen™ Molecular Probes™ CA, USA), which depends upon membrane potential for its accumulation. As is shown in FIG. 3D, almost all mitochondria were stained with both dyes, indicating intact membrane potential. Mitochondrial membrane potential (Δψₘ) is a key indicator of mitochondrial activity because it reflects the process of electron transport and oxidative phosphorylation, the driving force behind ATP production. Depolarization below a certain Δψₘ may indicate impaired mitochondrial function.

These results demonstrate successful isolation of pure and active mitochondria.

### Example 2

### In Vitro Uptake of Isolated Mitochondria by Various Cells

Having established a method for isolation of active mitochondria, fresh isolated mitochondria were tested for their ability to enter various cell types: human neuronal SH-SY5Y cells, and cell types of occular origin such as human adult retinal pigment epithelial cell line-19 (ARPE-19), human corneal endothelial cells (hCEC), and mouse RGC cells (661W). Mitochndia were isolayted from ARPE-19 cells or from mouse live as described in Materials and Method, and their vitality (oxygen consomption, membrane potential) and activity (ATP production) was assessed as desribed in Example 1 above. Mitochondria were labled with MTG for flow cytometry or with MTR for microscopy, and incubated with the cells for 24 h. For fluorescence microscopy, human SH-SY5Y neuronal cells were fixed and stained for the cytosolic protein β-actin, and for the endogenous mitochondrial protein Cox4.

Flow cytometry analysis demonstrated an uptake of mitochondria by the neuronal (SH-SY5Y) cells (FIG. 4A). Neurons transplanted with exogenous mitochondria showed an increase in fluorescence compared to non-stained negative control neurons. Neurons not transplanted with mitochondria and stained for their endogenous mitochondria with MTG served as a positive control, and unstained SH-SY5Y cells not transplanted with exogenous mitochondria served as negative control.

The uptake of mitochondria was also observed using fluorescence microscopy. Immunofluorescence staining shown in FIG. 4B demonstrated uptake of purified mitochondria stained with MTR (Mito; upper left image; red spots) by neuronal cells immunostained for β-actin to mark cytosolic, non-mitochondrial protein (Actin, upper middle image; green) and for Cox4 to mark endogenous mitochondria (Cox4, lower middle image; light blue), and further nuclear stained with DAPI (upper right image; blue). As shown in FIG. 4B, lower right image (Merge), MTR-stained exogenous mitochondria were found in the neurons cell body, indicating mitochondrial uptake. The arrow in the lower right panel shows exogenous mitochondria translocated into axons in a so-called “neuronal process”. A neuronal process refers to a projection from the cell body of a neuron that can be either an axon or a dendrite. Neuronal processes are characterized by localization of mitochondria in distal axons or dendrites, namely, long extensions far from the cell body, in order to increase neurons survival after injury. Three-dimensional viewing of confocal planes showed close localization of exogenous (red) and endogenous (yellow) mitochondria (FIG. 4C), confirming the internalization of exogenous mitochondria by the cells, in close proximity to endogenous mitochondria.

Other cell types of occular origin, including human ARPE-19, hCEC, and mouse RGC cells (661W) were able to internalize the exogenous mitochondria, as revealed by increased green fluorescence in flow cytometry following incubation with 1 million MTG stained mitochondria (“1M Mito” in FIG. 4D).

Incubation of hCEC and 661W cells with increasing amounts of mitochondria showed a dose-dependent uptake (FIGS. 5A-5C). These results demonstrate that cells from ocular and neuronal origin have the ability to internalize exogenous mitochondria.

### Example 3

### In Vitro ATP Production in Mitochondrial Transplanted Cells

Mitochondria were isolated from C57BL/6 mice livers and their vitality (oxygen consomption, membrane potential) and activity (ATP production) was assessed as desribed in Example 1 above. The freshly isolated mitochondria were incubated with mouse RGC 661W cell line for 24 h, and ATP produced by these mitochondria-transplanted cell was measured as described in Materials and Methods.

As seen in FIG. 6, mitochondrial uptake by 661W cells lead to increased ATP production in these cells. ATP levels were normalized to cell number (assessed by Neutral Red test) and presented relatively to control cells (i.e., cells not transplanted with exogenous mitochondria). The results were obtained from 5 independent experiments.

### Example 4

### In Vitro Mitochondrial Uptake Following Oxidative Stress

The therapeutic potential of mitochondrial transplantation against oxidative stress was assessed in vitro in mouse retinal ganglion cells (661W cells). Mitochondria, freshly isolated from C57BL/6 mice livers, were tested for activity and vitality and stained with MTG as described above. Oxidative stress was induced by applying H₂O₂. Four groups of 661W cells were tested: (i) cells not incubated with exogenous mitochondria and not treated with H₂O₂; (2) cells incubated with exogenous mitochondria for 24 h but not pre-treated with H₂O₂; (3) cells not incubated with exogenous mitochondria but treated with H₂O₂ for 1 hour; and (4) cells treated with H₂O₂ for 1 hour and then incubated with exogenous mitochondria for 24 h. Cells were treated with 0.75 or 1 mM H₂O₂. Exogenous mitochondrial uptake was assessed by flow cytometry.

As shown in FIG. 7, oxidative stress significantly enhanced mitochondrial uptake. No cell death was observed (n=2 from 2 independent experiments).

### Example 5

### In Vivo Retinal Uptake of Isolated Mitochondria

Having demonstrated neuronal uptake of exogenous mitochondria in vitro, the efficiency of exogenous mitochondria entrance into retinal ganglion cells (RGCs) in vivo was next examined. Purified mitochondria (10⁶-10⁷) isolated from mouse liver were labeled (stained) with MTR and injected at two concentrations into the vitreous of one eye of each mouse. The contralateral eye was injected with PBS and served as control. Twenty-four hours after mitochondria transplantation, mice were euthanized, and their eyes were enucleated and fixed. Wholemount retina was immunostained for ganglion cells marking by Tuj1 (neuron-specific class III β-tubulin), as described in Materials and Methods.

As shown in FIG. 8A, MTR-stained mitochondria (red) were present in the outer surface of the retina, in a dose-dependent manner. Confocal microscopy and immunostaining confirmed the entry of the transplanted mitochondria into RGCs (FIGS. 8B and 8C). It is noted that these results are the first in vivo demonstrations of the ability to effectively transplant mitochondria into the eye, and more specifically into RGCs.

### Example 6

### Mitochondrial Uptake by RGCs Following Optic Nerve Crush (ONC)

To evaluate, in vivo, the therapeutic potential of mitochondrial transplantation in retinal ischemic insult, an accepted mouse model of non-arteritic anterior ischemic optic neuropathy (NAION) was used. This model is the most common clinical presentation of acute ischemic damage to the optic nerve. Using this model, the right optic nerve was crushed by applying forceps at 2.5 to 3.0 mm posterior to the globe for 7 seconds; this procedure was repeated three times. Optic nerve crush was immediately followed by a single intravitreal injection of 10⁶-10⁷ isolated mitochondria stained with MTR. The contralateral (left) eye of each mouse, which was not damaged, was intravitreally injected with MTR-stained mitochondria. Mice were euthanized 24 h following mitochondrial transplantation, the eyes were enucleated and fixed. Wholemount retina were prepared, RGCs were immunostained and/or stained with DAPI as described in Materials and Methods and visualized by a fluorescent microscope. As shown in FIGS. 9A-9B, following ONC, mitochondrial uptake by retinal cells was enhanced.

The retina has a very organized structure with specific layers of cell bodies and fibers (see FIGS. 2A-2B). The inner most (and thinnest) cell layer is the ganglion cell layer (GCL). Paraffin-embedded sections of eyes of the-above-described NAION mice, stained with DAPI, were visualized by fluorescence microscope. As shown in FIG. 10, the injected mitochondria resided in the GCL. The enlarged frames 1 and 2 are zoom-in of the rectangular frames in the middle panel.

### Example 7

### The Effect of Mitochondrial Transplantation on Retinal on Injured Ganglion Cells

For demonstrating improved RGCs survival following ONC by way of mitochondrial uptake, RGCs were counted in sections of retinae obtained from NAION mice intravitreally injected with purified, 10⁷ isolated mitochondria immediately following ONC. One eye in the optic nerve crushed mouse was injected with mitochondria, and the contralateral eye was injected with PBS (control). Mice undergoing ONC without concomitant mitochondria injection served as a negative control. Two weeks after injection, eyes were harvested, sectioned and stained with hematoxylin & eosin (H&E) as described in Materials and Methods. Retinal ganglion cells were counted at ×40 magnification field.

Exemplary stained retinal sections are shown in FIGS. 11A-11C, demonstrating loss of RGCs following ONC, but rescue by mitochondria transplantation. The arrows point to the RGCs layer. Quantification of RGCs number in the indicated treatment protocols is presented in FIG. 11D.

Clearly, these results show that mitochondrial transplantation after ONC resulted in reduced number of RGCs death two weeks after injury compared to control.

### Example 8

### The Effect of Mitochondrial Transplantation on Oxidative-Stress Outcome

Oxidative stress was used as a model for ischemic injury in order to assess the ability of exogenous mitochondrial uptake to protect cells from ischemic stress-induced cell death. For this purpose, two human cell lines of occular origin were used: hCEC and ARPE-19. Cells were treated with H₂O₂ (hCEC cells with 2.5 μM, ARPE-19 cells with 250 μM H₂O₂) for one hour before transplantation with 100,000 mitochondria isolated from ARPE-19 cells. This setting further examined the protective effect of mitochondria both in a syngeneic system (genetically identical, i.e., ARPE-19 mitochondria transplanted in ARPE-19 cells) and allogeneic system (genetically different subjects of the same species, i.e., ARPE-19 cells mitochondria transplanted in hCEC cells). Eighteen hours after transplantation, cell survival was determined by XTT (ARPE-19 cells) or Neutral Red (hCEC cells) assays, both evaluating the number of viable cells.

As shown in FIGS. 12A-12B, mitochondrial transplantation increased survival of both cell types following oxidative stress. This is consistent with several reports demonstrating protective effect of allogeneic mitochondria in various animal models of ischemic injury. Furthermore, mitochondria uptake by hCEC isolated from the limbus of human corneas, suggests that mitochondria can be injected into the limbus for treating conditions where there is limbal stem cell deficiency.

### Example 9

### The Effect of Mitochondrial Transplantation on Corneal Wound Healing

For inducing alkali corneal injury to the eye, mice were anesthetized by intraperitoneal injection of ketamine (75 mg/g) and dormitory (0.75 mg/g). Localin (oxybuprocaine hydrochloride 0.4%) was topically applied and left on the eye for 1 minute. Alkali burns were inflicted in the right eye of mice as follow: Whatman 3 mm filter paper was cut into 5 mm diameter circles, soaked in 0.15 M NaOH for 4-5 seconds, and placed on the eye for 30 seconds. Then, the eye was extensively washed by 5 ml sterile PBS.

For mitochondria transplantation, isolated mitochondria obtained as described in Material and Methods, were diluted in IB-1 buffer and a 25 μl drop was applied to the alkali-injured eye. The drop was replenished every 15 minutes for a total of 3 hours. Finally, dethamycin (dexamethasone 21-phosphate disodium salt, 0.1%) and neomycin sulfate (0.5%) were topically applied to the injured eye. Dethamycin exhibits therapeutic effects, inter alia, against postoperative cataract. Celluspan (hydroxyethylcellulose 1.4%) was applied to non-treated eye to avoid drying. Anesthesia was reversed by subcutaneous injection of antisedan (antipamezole hydrochloride 3.75 mg/g). Mitochondria transplantation was performed daily from the day of injury and for 4 consecutive days. Control mice were treated with IB-1 buffer only and applied the same antibiotics and steroids as the mitochondria transplanted eye.

Every 24 hours mice were anesthetized as described in Materials and Methods, the cornea was stained with 0.1% fluorescein by topical administration and epithelial damage was evaluated using slit-lamp microscopy. The results are shown in FIGS. 13A-13B.

Slit-lamp images and fluorescein staining of alkali injured mouse epithelium is shown in FIG. 13A. As shown in the upper raw of images, alkali injury without treatment is not fully closed after 4 days, whereas eye treated with mitochondria transplantation presented full closer of the injury after 4 days.

Wound size was countified as the ratio between full eye area and damaged epithelial area (in percentages) from day 1 to day 4. In the eyes of the control group, the damage area closed by 40% in day 4, while in the mitochondria transplanted eyes, the damaged area was 1.5% in day 4. The epithelium wound size closure rate was faster after the addition of mitochondria to the injured eyes compared to the control group P<0.0001 (FIG. 13B).

Four days after injury, corneas were isolated and stained by H&E. The results are shown in FIG. 14A-14B: FIG. 14A shows representative H&E images of corneas harvested 4 days post alkali damage from mice receiving mitochondria transplantation (Mito) or vehicle as control (Control). Arrowhead denotes initial damage location. FIG. 14B shows central corneal thickness as determined from the mean of three measurements at the central region of each cornea histology section. n=3 mice (control); 3 mice (mito). p<0.01 (Student's T-test).

These results have important implications for the development of new treatments for corneal injuries caused by chemical agents like NaOH. The use of mitochondrial transplantation could offer a new and effective approach for promoting faster healing and reducing the risk of complications such as visual impairment.

### Example 10

### The Effect of Mitochondrial Transplantation on Proliferation and Wound Healing in Human Corneal Epithelial Cells

For assessing mitochondria transplantation into primary human corneal epithelial cells, primary human corneal epithelial cells were detached from human corneas and cultured. A scratch in the shape of a cross was performed on confluent cultures. Then, isolated mitochondria obtained from ARPE-19 cells as described in Material and Methods, were diluted in IB-1 buffer and incubated with the corneal epithelial cell cultures. Cultures were imaged and the wound area was measured by ImageJ at time 0, and 24 hours after scratching. The results are shown in FIGS. 15A-15B. Thus, as seen, while the mean percentage of wound closure was 60% in the control group (FIG. 15A, upper panel), mitochondria-transplanted cells demonstrated complete closure of the wound (100%) 24 hours post wounding (FIG. 15B, lower panel).

Primary human epithelial cells with or without mitochondria transplantation, as described above, were stained with antibodies against ki67, a marker for cell proliferation, or incubated with Neutral Red, a dye that is actively absorbed only by living cells. The results are shown in FIGS. 16A-16B. As seen, the percentage of ki67-positive cells was approximately 12% higher in mitochondria-transplanted cells, compared to control (FIG. 16A). Neutral Red uptake was approximately 17% higher, on average, in cells treated with mitochondria, compared to control cells (FIG. 16B).

These results demonstrate that mitochondria transplantation induces proliferation and wound healing in human corneal epithelial cells.

### Example 11

### The Effect of Exogenous Mitochondrial Transplantation on Corneal Endothelial Cell Migration

Migration of human corneal endothelial cells (hCEC-12 cell line) was assessed using a scratch wound assay. Briefly, a monolayer of cultured hCEC-12 was grown until it formed a continuous sheet. A cross shaped “wound” was then created by dragging a sterile pipette tip across the cell layer, thereby removing cells along the narrow bands of the cross pattern. To eliminate the contribution of cell proliferation, cultures were pretreated with mitomycin-C (10 μg/ml). After the scratch was made, cultures were washed to remove debris, and the cells were allowed to recover under defined experimental conditions (i.e., with or without mitochondria transplantation).

Isolated human mitochondria were applied to the media of confluent cultures immediately following scratching (Day 0) and remained in the media for 24 hours. Over time, the cells at the edges of the wound began to migrate into the gap. Wound closure was quantified by measuring the reduction in the scratch area using image analysis software. Images were taken at different time points to track how quickly and completely the wound area closed. The results are shown in FIGS. 17A-17B.

As shown in FIG. 17A, treatment with mitochondria enhanced cell migration compared to untreated controls indicating a pro-migratory effect, independent of proliferation, in human corneal endothelial cells. Quantification of the remaining wound area was analyzed by Student's t-test (FIG. 17B).

In a further ex vivo study, primary human corneal endothelial cells were isolated from harvested human corneas (rejected for corneal transplantation). Corneal endothelial cells were cultured and subjected to a scratch assay with or without supplementation of exogeneous mitochondria, as described above. Wound closure was measured daily for 4 days under light microscopy. Five individual experiments were conducted with 1 control and 1 test group in each. In each experiment cells were isolated from different corneas and treated with individual mitochondria isolations (5 million mitochondria per treatment). The results are shown in FIGS. 18A-18B.

The images presented in FIG. 18A demonstrate that mitochondria supplementation significantly enhanced wound closure compared to control after 4 days. The reduction in wound size (% of original size) was quantified in the graph presented in FIG. 18B. Mitochondria supplementation enhanced the migratory function of primary human corneal endothelial cells as in the in vitro scratch assay.

These results show that restoring mitochondrial function markedly enhanced endothelial migration and reduced cell-free areas in the monolayer following cell damage, suggesting that mitochondrial transplantation offers a strategy to promote corneal endothelial repair.

### Example 12

### The Effect of Exogenous Mitochondrial Transplantation on Corneal Endothelial Cell Density in Culture

Primary human corneal endothelial cells were isolated from harvested human corneas (rejected for corneal transplantation), cultured to form sparse cultures, and either treated with exogeneous mitochondria or not. Cell density was determined by counting cells per area. Two individual experiments were conducted, one control and one test group in each. Cells were isolated from different corneas and treated with individual mitochondria isolations (5 million mitochondria each). Forty randomly selected cell fields per sample were counted in each experiment on day 4.

The results shown in FIG. 19 indicate that mitochondrial supplementation increased endothelial cell density in sparse cultures of primary human CECs.

### Example 13

### The Effect of Exogenous Mitochondrial Transplantation on ATP Production in Human Corneal Endothelial Cells

HCEC-12 corneal endothelial cells were incubated with isolated mitochondria (purified from human ARPE-19 cells), or with growth media only as control, for 4 hours. Two individual controls and two individual mitochondria groups from two separate mitochondria isolations were employed. Then, the cells were harvested, and ATP content was measured using the ATPlite™ luminescence assay kit (as described in Materials and Methods) and read by a microplate reader.

The bar graph shown in FIG. 20 presents an increase in absorbance, reflecting higher cellular ATP levels measured in cells receiving mitochondria compared to control cells.

### Example 14

### The Effect of Exogenous Mitochondrial Transplantation on Oxidative Stress-Associated Cellular Reorganization of Human Corneal Endothelial Cells

Fuchs' endothelial corneal dystrophy (FECD) is a progressive, age-related disease of the cornea in which the endothelial cells, the cells responsible for pumping fluid out of the cornea, gradually deteriorate. As these cells are lost, the cornea retains excess fluid and becomes swollen, leading to visual impairment.

FECD is thought to be caused by a combination of genetic predisposition and environmental or age-related factors. Mutations in genes such as TCF4, COL8A2, and others are commonly implicated. The disease is characterized by the formation of abnormal collagen deposits on Descemet's membrane called guttae, which interfere with endothelial function. Oxidative stress and mitochondrial dysfunction are thought to contribute to endothelial cell loss, and oxidative stress is widely regarded as a key component of the pathophysiology of Fuchs' endothelial corneal dystrophy.

Main symptoms include blurred or cloudy vision, often worse in the morning; glare and light sensitivity; fluctuating vision throughout the day; and halos around lights. In advanced stages, pain or foreign-body sensation due to epithelial blisters (bullae) from severe corneal swelling

Overall, FECD leads to progressive loss of transparency in the cornea, ultimately impairing vision and sometimes requiring corneal transplantation in advanced cases.

Human CEC-12 cells were exposed to 15 μM menadione to induce oxidative stress and generate guttae-like cellular clusters (also referred to here as “rosettes”), a phenotype reminiscent of early pathological changes observed in FECD. Cells treated with DMSO served as solvent-only controls. To minimize proliferation and isolate the effects of oxidative stress and mitochondrial rescue, all treatments were performed for 2 hours at 37° C. under serum-free conditions. Following this exposure, menadione was removed and replaced with fresh serum-free medium either supplemented with isolated mitochondria (20 million, human ARPE-19 source) or without added mitochondria (control). Cells were then incubated for an additional 24 hours to allow for potential structural recovery.

After 24 hours, cultures were imaged, and the areas of rosette-like clusters were quantified. The results are shown in FIGS. 21A-21B.

As shown in FIG. 21A, mitochondrial supplementation significantly reduced the extent of rosettes clusters. The decrease in the rosette area was approximately 33% compared with menadione-treated controls (FIG. 21B). This finding indicates that restoring mitochondrial function can mitigate oxidative stress-induced structural abnormalities characteristic of corneal endothelial dysfunction.

### Example 15

### The Effect of Exogenous Mitochondrial Transplantation on ERK and JNK MAPK Signaling in Human Corneal Endothelial Cells

Extracellular signal-regulated kinase (ERK) and c-Jun N-terminal kinase (JNK) are major components of the mitogen-activated protein kinase (MAPK) family and are known to mediate processes such as cell survival, proliferation, apoptosis, response to oxidative stress and migration. Because mitochondrial dysfunction and oxidative injury can directly modulate MAPK signaling, assessing ERK and JNK activity provides important insight into how exogenous mitochondria influences stress-related signaling cascades. Thus, the effect of mitochondrial transplantation on the ERK and JNK signaling pathways was assessed in hCEC-12 cells.

Human CEC-12 were incubated for three hours with either isolated mitochondria or medium alone (control), and protein lysates were analyzed by Western blot for total and phosphorylated active ERK and JNK (P-ERK and P-JNK). ERK and JNK were detected using well-validated antibodies that recognize either the total protein (regardless of activation state) or the phosphorylated, active form (phospho-specific antibodies). Both total and phospho-specific antibodies were used for blotting since they allow normalization and assessment of activation independent of expression level. The results are shown on FIG. 22.

As seen, although total ERK and JNK levels were similar between groups, the phosphorylated active forms were markedly elevated in the mitochondria-treated cells. These findings suggest that transplantation of exogenous mitochondria enhances activation of the ERK and JNK pathways, potentially contributing to improved cellular responses to oxidative stress.

### Example 16

The Effect of ERK and JNK Activation On Mitochondria-Induced Migration in hCEC-12 Cells

Based on the observation that exogenous mitochondria enhance activation of the ERK and JNK pathways in hCEC-12 cells, the functional contribution of these signaling cascades to the pro-regenerative effects of mitochondrial transplantation was evaluated.

Human CEC-12 cell monolayers were mechanically wounded (scratched), and the initial wound areas were documented by imaging, as described in Example 11 above. Cells were then pre-treated for 1 hour with either MEK (the kinase activating ERK) or JNK inhibitors, both inhibitors together, or DMSO as solvent control. U-0126, a well-known MEK1/2 inhibitor (10 μM) was used for blocking the ERK signaling pathway, and SP600125, an ATP-competitive inhibitor of JNK1, JNK2, and JNK3 (40 μM) was used for blocking JNK.

Following inhibitor pre-treatment, the culture media (containing the respective inhibitors) was either supplemented with exogeneous isolated mitochondria (about 20 million) or not. After 24 hours, cultures were re-imaged and wound areas were quantified using image-analysis software. The results are shown in FIGS. 23A-23B.

Mitochondrial transplantation significantly accelerated wound closure (FIG. 23A), reducing the cell-free wound area by approximately 23% relative to control cultures (FIG. 23B). However, pharmacologic inhibition of either MEK or JNK markedly blunted this effect, impairing wound closure by 36% and 54%, respectively, compared to DMSO-treated, mitochondria-supplemented cells. Co-inhibition of MEK and JNK produced an effect comparable to JNK inhibition alone, increasing residual wound area by 52%.

Together, these findings demonstrate that activation of the ERK and JNK pathways plays a critical role in mediating the enhanced wound-healing response elicited by mitochondrial transplantation in corneal endothelial cells. This suggests that the mitochondrial effect in corneal wound healing will be augmented or improved by co-application of one or more modulators of MAPK pathways, particularly agents or substances that facilitate, activate or promote MAPK pathways.

### Example 17

### The Effect of Mitochondria Transplantation on Corneal Edema or Thickness

Corneal injury was induced in Sprague-Dawley rats using a standardized transcorneal cryo-damage protocol, in which a metal probe cooled in liquid nitrogen was applied to the central cornea to create controlled endothelial injury. The resulting selective damage to the corneal endothelium was previously verified by the present inventors using alizarin red staining, which highlights endothelial loss and morphological disruption.

Immediately after injury, animals received an intracameral injection of about 10 million mitochondria, i.e., the treatment solution was delivered directly into the anterior chamber of the eye, the fluid-filled space between the cornea and iris that lies in immediate contact with the corneal endothelium. This route enables precise, localized delivery. Each rat was injected with either isolated mitochondria or PBS as a control. The results are shown in FIG. 24.

Corneal thickness, a sensitive clinical indicator of corneal endothelial dysfunction and stromal edema, was then monitored using anterior-segment optical coherence tomography (OCT). As expected, injury-induced endothelial impairment led to increased corneal swelling in control eyes. In contrast, mitochondrial treatment significantly reduced corneal thickness, indicating improved endothelial function. These findings demonstrate that mitochondrial transplantation can mitigate or repair corneal endothelial damage, supporting its therapeutic potential in corneal injury and endothelial disorders.

### Example 18

### Mitochondria Transplantation for Treatment of Retinal Degeneration

First, tolerance of mice to repeated intravitreal injections of mitochondria was assessed.

Mitochondria were isolated from livers of C57BL/6 mice and injected in increasing doses (0.5×10⁵, 2×10⁵, 1×10⁶) to the vitreous of C57BL/6 mice every two weeks, for a total of twelve weeks. Control mice were injected with PBS only. Ocular safety was assessed by serial optical coherence tomography (OCT) and fundus imaging. No structural abnormalities, retinal damage or inflammation following repeated administration were observed, as shown in FIG. 25A. Immunostaining for class III β-tubulin (Tuj1) shown in FIG. 25B, demonstrated preserved retinal ganglion cell integrity, indicating no evidence of cell loss or neurotoxicity.

Then, the ability of mitochondria transplantation to treat diabetic retinopathy was assessed. Diabetes was induced in C57B1/6 mice by streptozotocin administration. In the test group, isolated mitochondria or PBS were administered by intravitreal injection every two weeks, starting at the onset of diabetes (>250 mg/dL blood glucose). Mice which were not treated with streptozotocin (i.e., healthy mice) served as control.

Retinal ganglion cells (RGC) thickness and neural retina thickness are two different structural measurements within the retina, each capturing a different level of anatomical detail. RGC thickness (also herein termed “retinal ganglion cell layer thickness” or “GCL thickness”) measures only the layer of the retina that contains the cell bodies of retinal ganglion cells layer (plus sometimes inner plexiform layer, depending on the method). These are the neurons that collect visual information from the eye and send it to the brain via the optic nerve. Because this is a single, specific retinal layer, its thickness is relatively small and reflects changes specifically affecting RGC health such as glaucoma-related loss of ganglion cells.

Neural retina thickness, in contrast, measures the thickness of all retinal layers collectively (i.e., the entire neurosensory retina), excluding only the retinal pigment epithelium (RPE) and choroid, namely, all layers above the RPE. It therefore represents the full layered architecture of the retina, from the inner limiting membrane (ILM) to the outer nuclear or outer segment region, depending on the measurement definition. This thickness reflects global retinal integrity rather than changes in a single cell population. Thus, RGC thickness is a specific, localized metric of ganglion cell health, whereas neural retina thickness is a broader measure of overall retinal structure.

The thickness of the retinal ganglion cell layer and the neural retina layer was monitored by OCT imaging 13 weeks post onset of diabetes. ImageJ (an image-analysis program) was used to measure the thickness of the RGC layer as well as the entire neurosensory retina, and each measurement was expressed as a percentage of the average RGC/neural retina layer thickness of the healthy control mice. This allowed easier comparison between treated and untreated groups. Analysis of variance (ANOVA) was the statistical test applied to determine whether there were significant differences in RGC layer thickness between the different experimental groups.

The results are shown in FIGS. 26A-26B. As shown, mitochondria injected eyes exhibited reduced retinal thinning compared to diabetic controls injected with phosphate-buffered saline, indicating a protective effect of transplanted mitochondria on retinal structure.

What is claimed is:

1. A method for treatment of damaged endothelium of the cornea, comprising transplanting a therapeutically effective amount of exogenous, isolated mitochondria to the damaged corneal endothelium cells thereby treating the damaged endothelium of the cornea.

2. The method of claim 1, wherein damage to the corneal endothelium is caused by one or more of: oxidative stress, age-related decline in endothelial cell density, inherited or non-inherited degenerative disease, surgical trauma, injury or ocular trauma, inflammation, infection, increased intraocular pressure (IOP), contact lens-related hypoxia, contact lens overuse, a corrosive agent, exposure to a chemical or toxin, chemical or thermal burns, reduced oxygen or nutrient supply to the cornea, a systemic disease and cryotherapy or laser procedures.

3. The method of claim 2, wherein the corneal endothelium damage is caused by oxidative stress.

4. The method of claim 2, wherein the corneal endothelium damage is caused by Fuchs endothelial corneal dystrophy.

5. The method of claim 1, wherein the mitochondria are transplanted via a local route, a systemic route or a combination thereof.

6. The method of claim 5, wherein mitochondrial transplantation is via at least one route selected from the group consisting of topical application, cell-mediated transfer, vesicle-mediated delivery, nanoparticle/polymer mediated delivery, peptide-mediated delivery, subconjunctival injection or direct injection and microinjection to the eye.

7. The method of claim 1, wherein the mitochondria are freshly isolated mitochondria, preserved mitochondria or a combination thereof.

8. The method of claim 1, wherein the mitochondria are least on of autologous, allogeneic, syngeneic or xenogeneic.

9. The method of claim 1, wherein the therapeutically effective amount of mitochondria is in the range of from about 10⁴ to about 10¹⁰ mitochondrial dose per administration.

10. The method of claim 9, wherein the therapeutically effective amount of mitochondria is in the range of from about 10⁶ to about 10⁷ mitochondrial dose per administration.

11. The method of claim 1 further comprising the administration of one or more modulators of MAPK pathways or ROCK pathways.

12. A method for treating retinal degeneration, comprising transplanting a therapeutically effective amount of exogenous, isolated mitochondria to the retinal layer, thereby treating the degenerated retina.

13. The method of claim 12, wherein the exogenous, isolated mitochondria are transplanted into one or more of: degenerated photoreceptors, degenerated retinal ganglion cells, and degenerated retinal pigment epithelium (RPE) cells.

14. The method of claim 12, wherein treatment comprises prevention, early intervention, slowing progression, and ameliorating, improving or curing advanced or late-stage retinal degenerative disease.

15. The method of claim 12, wherein the retinal degeneration is not directly associated with mitochondrial dysfunction.

16. The method of claim 12, wherein the retinal degeneration is caused by one or more of: an inherited retinal disorder, age-related degenerative process, metabolic disease or disorder, vascular disease or disorder, ischemic insult, chronic inflammation, immune-mediated injury or disease, infectious, traumatic or toxic insult, an inherited retinal dystrophy, light-induced oxidative stress, drug-induced retinal degeneration, nutrient deficiencies, or loss of retinal ganglion cells and retinal thickness

17. The method of claim 16, wherein the retinal degeneration is caused by diabetic retinopathy or glaucoma.

18. The method of claim 12, wherein mitochondria are transplanted via one or more routes selected from subretinal injection, intravenous injection, topical application, cell-mediated transfer, vesicle-mediated delivery, nanoparticle/polymer mediated delivery, peptide-mediated delivery, subconjunctival injection or direct injection or microinjection to the eye.

19. The method of claim 12, wherein the mitochondria are freshly isolated mitochondria, preserved mitochondria or a combination thereof, the mitochondria being at least one of autologous, allogeneic, syngeneic or xenogeneic mitochondria.

20. The method of claim 12, combined with anti-VEGF therapy.

21. A method for preventing or treating diabetic retinopathy in a subject, comprising transplanting a therapeutically effective amount of exogenous, isolated mitochondria to the retina of the subject, thereby preventing or treating diabetic retinopathy in the subject.
